(19) United States
(12) Patent Application Publication
(10) Pub. No.: US 2023/0287038 A1
(43) Pub. Date: Sep. 14, 2023

(54) PURIFIED SAPONINS AND CHROMATOGRAPHIC PROCESS FOR PURIFICATION OF SAME

(71) Applicant: ACCESS TO ADVANCED HEALTH INSTITUTE, Seattle, WA (US)

(72) Inventors: Yizhi QI, Seattle, WA (US); Christopher Bradford FOX, Sumner, WA (US)

(21) Appl. No.: 18/020,253

(22) PCT Filed: Jun. 4, 2021
(86) PCT No.: PCT/US21/36015

§ 371 Date: Feb. 7, 2023

Related U.S. Application Data
(60) Provisional application No. 63/106,752, filed on Oct. 28, 2020.
(60) Provisional application No. 63/063,121, filed on Aug. 7, 2020.

(51) Int. Cl. C07J 63/00; A61K 39/39; G01N 30/86; G01N 30/72; B01D 15/30; B01D 15/32

(52) U.S. Cl. CPC C07J 63/008; A61K 39/39; G01N 30/8631; G01N 30/72; B01D 15/305; B01D 15/325; A61K 2039/55577

(57) ABSTRACT

Substantially pure saponin extracts and orthogonal chromatographic methods for purification of saponin extracts are disclosed. The purified saponin extracts may include QS-21 and can have a purity of greater than 97%. The orthogonal chromatographic method uses reversed-phase (RP) chromatography followed by hydrophilic interaction liquid chromatography (HILIC) to generate substantially pure saponin extracts.

### CROSS-REFERENCE TO RELATED APPLICATION

This application claims priority to U.S. Provisional Patent Application No. 63/063,121, entitled “Purified Saponins and Chromatographic Process for Purification of Same,” filed on Aug. 7, 2020, and U.S. Provisional Patent Application No. 63/106,752, entitled “Purified Saponins and Chromatographic Process for Purification of Same,” filed on Oct. 28, 2020, the disclosures of which are incorporated herein by reference in their entireties.

### STATEMENT REGARDING FEDERALLY SPONSORED RESEARCH OR DEVELOPMENT

This invention was made with government support under Contract No. HHSN272201300029C awarded by National Institute of Allergy and Infectious Diseases, National Institutes of Health, and Department of Health and Human Services. The government has certain rights in the invention.

### TECHNICAL FIELD

This present disclosure relates generally to techniques for chromatographic purification of plant extracts. Specifically, this disclosure provides a technique for processing Quillaja saponaria extracts to obtain substantially pure QS-21 or other saponins.

### BACKGROUND

Adjuvants are compounds that help enhance immune response to vaccine antigens. Adjuvants are included in vaccines to improve humoral and/or cellular immune responses, particularly in the case of poorly immunogenic subunit vaccines. Similar to natural infections by pathogens, adjuvants rely on the activation of the innate immune system to promote long-lasting adaptive immunity.

Saponins extracted from the bark of the Chilean soap bark tree Quillaja saponaria are well-known adjuvants. (Ragupathi et al., Expert Review of Vaccines, Vol. 10(4), 2011, pages 463-470; C. R. Kensil, QS-21 adjuvant, in: D. T. O'Hagan (Ed.) Vaccine Adjuvants, Humana Press, Totowa, N.J., 2000, pp. 259-271). Extracts from the bark of Quillaja saponaria contain a mixture of triterpene glycosides. Crude saponin extracts have limited usability as adjuvants because of the presence of impurities, toxins, and variation in adjuvant activity.

There are various known methods for the purification of saponins, including solvent extraction, adsorption, ultrafiltration, and chromatography. For example, US Patent Application Publication No. 2014/0030318 describes a method for purification of saponins using solubilizing compounds and exchange solvents, followed by dilution or dialysis. Chilean Patent Application No. CL 200202573 discloses a process for production of saponins by elimination of impurities with adsorbents followed by filtration. Similarly, U.S. Pat. No. 6,355,249 describes purification of saponins by ultrafiltration and high performance liquid chromatography. Saponins have also been extracted through a series of salting and filtration steps as described in WO 2017/091333.

Early work using reversed-phase chromatography to purity saponin extracts identified at least 22 separate peaks. (U.S. Pat. No. 5,057,540; C. R. Kensil, U. Patel, M. Lennick, D. Marciani, Separation and characterization of saponins with adjuvant activity from Quillaja saponaria Molina cortex, J. Immunol., 146 (1991) 431-437). Four fractions referred to as QS-7, QS-17, QS-18, and QS-21 were identified with adjuvant activity. QS-18 was found to be toxic and QS-7 was less abundant than QS-21. The QS-21 fraction isolated from extracts of Quillaja saponaria bark has been widely studied as a saponin adjuvant. The mass/charge ratios (m/z) of singly deprotonated ([M-H]⁻¹) QS-7, QS-17, QS-18, and QS-21 detected by Liquid Chromatography/Mass Spectrometry (LC/MS) have been previously reported as 1862, 2296, 2150, and 1988 respectively. (H. L. Pham, B. P. Ross, R. R. McGeary, P. N. Shaw, A. K. Hewavitharana, N. M. Davies, Saponins from Quillaja saponaria Molina: Isolation, Characterization and Ability to Form Immuno Stimulatory Complexes (ISCOMs), Current Drug Delivery, 3 (2006) 389-397.)

QS-21 was initially identified as a particular chromatographic fraction from the extract of Quillaja saponaria bark that included strong adjuvant activity and favorable toxicity relative to other fractions. (C. R. Kensil, U. Patel, M. Lennick, D. Marciani, Separation and characterization of saponins with adjuvant activity from Quillaja saponaria Molina cortex, J. Immunol., 146 (1991) 431-437.) Subsequent characterizations identified the principle constituent of this particular fraction as an acylated triterpene glycoside with a molecular formula of C₉₂O₄₆H₁₄₈ and a molecular weight of 1,990 Daltons. (N. E. Jacobsen, W. J. Fairbrother, C. R. Kensil, A. Lim, D. A. Wheeler, M. F. Powell, Structure of the saponin adjuvant QS-21 and its base-catalyzed isomerization product by 1H and natural abundance 13C NMR spectroscopy, Carbohydr. Res., 280 (1996) 1-14; C. R. Kensil, S. Soltysik, U. Patel, D. J. Marciani, Structure/function relationship in adjuvants from Quillaja saponaria Molina, in: F. Brown, R. M. Chanok, H. S. Ginsberg, R. A. Lenner (Eds.) Vaccines 92, Cold Spring Harbor, Cold Spring Harbor, N.Y., 1992, pp. 35-40.)

QS-21 is an amphiphilic molecule consisting of a central quillaic acid triterpene core, flanked on either side by a branched trisaccharide and a linear tetrasaccharide, which is in turn connected to a fatty acyl chain. The trisaccharide attached at the C3-position of the quillaic acid comprises D-glucuronic acid, D-galactose, and D-xylose. The tetrasaccharide connected via the C28 carboxylate on the triterpene is a linear chain of D-fucose, L-rhamnose and D-xylose linked to one of two isomeric sugars, D-apiose or D-xylose, giving rise to two compositional isomers denoted as QS-21ₐₚᵢ (65%) and QS-21_{xyl} (35%), respectively. (U.S. Pat. No. 5,583,112; S. Soltysik, D. A. Bedore, C. R. Kensil, Adjuvant Activity of QS-21 Isomers, Ann. N. Y. Acad. Sci., (1993) 392-395.)

Additionally, an L-arabinose-terminated acyl chain is attached to the fucose residue via a hydrolytically labile ester bond. Intramolecular trans-esterification of this bond between the 4- and 3-hydroxyl groups on the fucose ring occurs naturally in solution, resulting in two regioisomers, QS-21A and QS-21B at a ratio of 20:1. (N. E. Jacobsen, W. J. Fairbrother, C. R. Kensil, A. Lim, D. A. Wheeler, M. F. Powell, Structure of the saponin adjuvant QS-21 and its base-catalyzed isomerization product by 1H and natural abundance 13C NMR spectroscopy, Carbohydr. Res., 280 (1996) 1-14; J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28; C. Pedebos, L. Pol-Fachin, R. Pons, C. V. Teixeira, H. Verli, Atomic model and micelle dynamics of QS-21 saponin, Molecules, 19 (2014) 3744-3760.) Therefore, QS-21 is not a single molecular entity but rather consists of four isomeric forms, all of which have previously been isolated and found to possess comparable adjuvant activity. (U.S. Pat. No. 5,583,112; S. Soltysik, D. A. Bedore, C. R. Kensil, Adjuvant Activity of QS-21 Isomers, Ann. N. Y. Acad. Sci., (1993) 392-395; J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28).

The success of QS-21 as a vaccine adjuvant is demonstrated by the vaccines Shingrix® and Mosquirix®. Shingrix® is a shingles vaccine with high efficacy even in older subjects that received regulatory approval in 2017. (U.S. Pat. No. 7,939,084). Mosquirix® is the first malaria vaccine and received regulatory approval in 2015. (WO 2017/102737). The specific adjuvant formulation used in the Shingrix® and Mosquirix® vaccines is described in A. M. Didierlaurent, B. Laupéze, A. D. Pasquale, N. Hergli, C. Collignon, N. Garçon, Adjuvant system AS01: helping to overcome the challenges of modern vaccines, Expert Rev. Vaccines, 16 (2017) 55-63 and A. M. Didierlaurent, A. Berger, T. C. Heineman, V. Henderickx, F. T. D. Silva, J. Vekemans, G. Voss, N. Garçon, The development of the adjuvant system AS01: a combination of two immunostimulants MPL and QS-21 in liposomes, in: Immunopotentiators in Modern Vaccines, Academic Press, 2017, pp. 265-285. A discussion of the mechanism of action of QS-21 in vaccines is provided in M.-A. Lacaille-Dubois, Updated insights into the mechanism of action and clinical profile of the immunoadjuvant QS-21: A review, Phytomedicine, 60 (2019).

The challenges of purifying specific saponins from a complex natural mixture containing a large number of impurities that are chemically very similar to the molecule of interest are well-known. (G. C. Kite, M.-J. R. Howes, M. S. J. Simmonds, Metabolomic analysis of saponins in crude extracts of Quillaja saponaria by liquid chromatography/mass spectrometry for product authentication, Rapid Commun. Mass Spectrom., 18 (2004) 2859-2870; Y. Wang, X. Lu, G. Xu, Development of a comprehensive two-dimensional hydrophilic interaction chromatography/quadrupole time-of-flight mass spectrometry system and its application in separation and identification of saponins from Quillaja saponaria, J. Chromatogr. A, 1181 (2008) 51-59; D. van Setton, G. van de Werken, G. Zomer, G. F. A. Kersten, Glycosyl Compositions and Structural Characteristics of the Potential Immuno-adjuvant Active Saponins in the Quillaja saponaria Molina Extract Quil A, Rapid Commun. Mass Spectrom., 9 (1995) 660-666.) Current processes for purifying saponin extracts are complex and limited by both yield and purity.

U.S. Pat. No. 5,057,540 describes a method for purifying QS-21 from a methanol extract of Quil-A® (a commercially-available saponin extract). This method uses silica chromatography followed by reversed-phase chromatography on a C4 column. Although the collected QS-21 fraction appeared as a single band in thin layer chromatography (TLC), Fast Atom Bombardment Mass Spectroscopy (FAB-MS) analysis run in negative ion mode showed that it was still a complex mixture. The mass spectrum shown for the QS-21 fraction (FIG. 8C) has prominent peaks at mass-to-charge ratios (m/z) of 895, 1314, and 1476. The peak at m/z 1988 represents QS-21 but at a relatively low intensity. The very noisy baseline further suggests the compositional complexity of this fraction.

A similar method is described in L. Brunner, C. Barnier-Quer, N. Collin, QS-21 Adjuvant: Laboratory-Scale Purification Method and Formulation Into Liposomes, in: C. B. Fox (Ed.) Vaccine Adjuvants, Springer Nature, New York, N.Y., 2017, pp. 73-86. This method processes Quil-A® with silica chromatography on an in-house packed column followed by reversed-phase chromatography on a C18 column. The purified QS-21 appears as a single peak with a leading shoulder on an analytical C18 column, though electrospray-ionization quadrupole time-of-flight mass spectrometry analysis shows a considerable number of impurity ions. The yield of purified QS-21 with respect to the weight of Quil-A® was reported to be −2%.

Another method that also uses a combination of silica and reversed-phase chromatography is described in U.S. Pat. No. 6,231,859. With this method, 20 g of a dialyzed and lyophilized aqueous Quillaja saponaria bark extract are separated on a silica column to obtain 3.2 g of intermediate product of approximately 51% purity. Next, 16 consecutive runs each with 20 mg of the intermediate product are performed on a reversed-phase C18 column. This is followed by a final capture and release step on a larger particle size C18 column to yield 59 mg of purified QS-21. The reported yield is 2.95% with respect to the starting extract. However, reported yields are difficult to compare directly because the various starting extracts likely contain differing amounts of QS-21.

The same U.S. Pat. No. 6,231,859 also describes a second approach for purification of saponin extracts. Polyvinylpolypyrrolidone (PVPP) is used to remove the polyphenolics from an aqueous bark extract of Quillaja saponaria. This is followed by sequential high-performance liquid chromatography (HPLC) runs on a reversed-phase phenyl column and a final capture and release step on a reversed-phase C8 column. Purity of both methods described in U.S. Pat. No. 6,231,859 are reported to be greater than 98% by reversed-phase HPLC. However, there is no chromatogram shown to support the purity claims. FAB-MS characterization of the final purified product (FIG. 2A) showing a limited m/z range of 1,500-2,200 revealed three primary impurities at negative ion m/z of 1856, 1922, and about 1950 besides the predominant QS-21 ion at m/z 1988. This patent also describes performance of consecutive repeated runs to produce the final purified product.

International Patent Application No. WO 2019/106192 describes a process for purifying QS-21 from a crude aqueous extract of Quillaja saponaria. The extract is first treated by polyvinylpyrollidone adsorption followed by diafiltration, ultrafiltration, or dialysis to clean the treated extract. Next, reversed-phase chromatography using a polystyrene column produces an intermediate product with a reported QS-21 content of greater than or equal to 18%. Additional chromatography step is performed using a reversed-phase phenyl column and a reversed-phase C8 column to concentrate the purified material before lyophilization. Final yield information is not provided.

Ultra-high performance liquid chromatography ultraviolet/mass spectrometry (UPLC-UV/MS) characterization shows that the purified QS-21 is at least 93% “QS-21 main peak” (containing triterpenoid glycosides having negative ion m/z of 1855.9, 1987.9, or 2001.9, excluding B-regioisomer, and a lyophilization impurity resulting putatively from the loss of the terminal D-apiose or D-xylose) and at least 98% “QS-21 group” (containing triterpenoid glycosides having negative ion m/z of 1517.7, 1711.8, 1855.9, 1987.9, 2001.9, 2017.9, or 2118, excluding the lyophilization impurity). Ions with m/z of 1517.7, 1711.8, and 2017.9 appear as separate impurity peaks in LC, with 2017.9 (2018) being the most dominant. This process is unable to remove the impurity with an m/z of 2018. Much of the disclosure is concerned with defining an acceptable range of m/z 2018 impurity in the starting extract. This limits the applicability of the process to purification of only Quillaja saponaria extracts with suitable amounts of the m/z 2018 impurity.

Isolation of substantially pure saponins from extracts of Quillaja saponaria is desirable to produce vaccine adjuvants. Current technologies are complex involving multiple steps such as non-chromatographic pre-treatment of the starting extract and multiple consecutive chromatographic separations and may require starting materials with specific characteristics. Moreover, the purified products produced by existing processes still include multiple impurities detectable with mass spectroscopy. Accordingly, there is a need for methods to purify extracts of Quillaja saponaria that are not complex and produce substantially pure saponins at high yield. The present disclosure fulfills these needs and offers other related advantages.

### SUMMARY

In one aspect, the present disclosure is directed to a substantially pure saponin extract comprising QS-21. In another aspect, the present disclosure is directed to an orthogonal chromatographic process for purification of saponins from Quillaja saponaria.

The present disclosure also provides pharmaceutical compositions and vaccine compositions containing saponin extracts. Additionally, the present disclosure describes use of a substantially pure saponin extract in the manufacture of a medicament and a method of eliciting or enhancing an immune response in a subject using a substantially pure saponin extract.

In one aspect, the substantially pure saponin extract is derived from Quillaja saponaria, contains QS-21, and is characterized by negative ion mass spectrum indicating a most abundant species at mass-to-charge ratio (m/z) 1989 and impurities at m/z 1857, 1923, and 2480. In an implementation, the substantially pure saponin extract is characterized by the absence of a mass spectral peak at m/z 2018. In an implementation, the substantially pure saponin extract is characterized by the absence of other impurity mass spectral peaks between m/z 750-2500 with an intensity greater than one-third the intensity of the m/z 1989 peak. In an implementation, substantially pure saponin extract is characterized by adjuvant activity.

The purity of the substantially pure saponin extract may be at least 95%, at least 96%, or at least 97%. Purity of the substantially pure saponin extract may be measured by percent area under the curve (AUC) of an analytical chromatography ultraviolet (UV) trace. In an implementation, the UV trace is characterized by a minor peak preceding a major peak and purity is measured by the percent AUC of the major peak. In an implementation, the UV trace uses light with a wavelength of 210 nm. In an implementation, the chromatography column is a traditional reversed-phase C4 column. In an implementation, the chromatography column comprises a butyl-functionalized silica solid phase and a water/acetonitrile mobile phase. The mobile phase may additionally include a volatile modifier.

The present disclosure provides a method of saponin purification that uses reversed-phase (RP) chromatography followed by hydrophilic interaction liquid chromatography (HILIC) to create a substantially pure saponin extract. The RP chromatography may use a traditional RP column or a polar RP column (“polar RP chromatography”). A partially enriched Quillaja saponaria extract may be purified by RP chromatography on a traditional RP chromatography column or by polar RP chromatography on a polar RP column that contains both nonpolar and polar moieties on the solid phase to create an intermediate purity saponin extract.

The partially enriched Quillaja saponaria extract may be a commercially available product such as VET-SAP® or Quil-A® or may be created by any suitable technique for processing the bark of Quillaja saponaria to extract saponins. In an implementation, the intermediate purity saponin extract is a fraction collected from the polar RP chromatography containing QS-21 and characterized by negative ion mass spectrum indicating a most abundant species above m/z 1200 among singly charged, intact saponins at m/z 1989. In an implementation, the intermediate purity saponin extract is a fraction collected from the polar RP chromatography containing QS-7 and characterized by negative ion mass spectrum indicating a most abundant species above m/z 1200 among singly charged, intact saponins at m/z 1862. In an implementation, the intermediate purity saponin extract is a fraction collected from the polar RP chromatography containing QS-17 and characterized by negative ion mass spectrum indicating a most abundant species above m/z 1200 among singly charged, intact saponins at m/z 2296. In an implementation, the intermediate purity saponin extract is a fraction collected from the polar RP chromatography containing QS-18 and characterized by negative ion mass spectrum indicating a most abundant species above m/z 1200 among singly charged, intact saponins at m/z 2150. The fraction may be collected so that it excludes any shoulder peaks. In an implementation, the intermediate purity saponin extract is dried by removal of solvent and then resuspended prior to purification by HILIC chromatography.

In an implementation, the traditional RP chromatography may be performed using a C18 column. In an implementation, the polar RP chromatography may be performed using a polar C18 column. The traditional RP chromatography and/or the polar RP chromatography may use a solid phase that is comprised of a material such as silica, ceramic, or polymer. The solid phase is functionalized with non-polar alkyl or aryl groups. For polar RP chromatography, the solid phase also includes polar groups that may be end-capped to free silanols or embedded within the alkyl or aryl groups. In an implementation, the polar RP chromatography may be performed using a solid phase with a pore size of about 60-300 Å. In an implementation, the polar RP chromatography may be performed using a mobile phase of water and an organic solvent. The organic solvent may be acetonitrile, methanol, tetrahydrofuran, ethanol, or isopropanol. In one implementation, the organic solvent is acetonitrile. In an implementation, the mobile phase includes a volatile modifier. The volatile modifier may be formic acid, trifluoroacetic acid (TFA), or acetic acid. The polar RP chromatography may be performed under gradient conditions (continuous or stepped) or isocratic conditions. In an implementation, the main separation gradient is 20-80% organic solvent. In an implementation, the main separation gradient is 30-70% organic solvent. In an implementation, the main separation gradient is 40-50% organic solvent.

The intermediate purity saponin extract collected following polar RP chromatography is further purified by HILIC to create a substantially pure saponin extract. The HILIC may use a solid phase that is comprised of unbonded (“bare”) or surface-functionalized silica. The solid phase may alternatively be a ceramic or a polymer. Polymers suitable for use in solid phase of a HILIC or other HPLC column include poly(styrene divinylbenzene), poly(vinyl alcohols), poly(acrylamides), poly(saccharides) and poly(methacrylates). In some implementations, the solid phase is surface-functionalized with diol functional groups, amide groups, amino groups, amide groups and amino groups, or zwitterionic groups. In an implementation, the pore size of the solid phase is about 70-200 Å. In an implementation, a mobile phase used in the HILIC includes water and an organic solvent. The organic solvent may be acetonitrile, acetone, methanol, or isopropanol. In one implementation, the organic solvent is acetonitrile. In an implementation, the mobile phase additionally includes a volatile salt. The volatile salt may be ammonium acetate or ammonium formate. The HILIC may be performed under gradient conditions or isocratic conditions. In an implementation, the mobile phase under isocratic conditions includes about 10%, about 20%, about 30%, about 40%, or about 50% water. In an implementation, the mobile phase under gradient conditions includes between about 10-50% water. In various implementations, the HILIC is performed at about pH 5-6 or about pH 5.8.

The substantially pure saponin extract may be any of QS-7, QS-17, QS-18, or QS-21. In an implementation, the substantially pure saponin extract collected from HILIC may be dried by removing the solvent to produce a dried saponin extract. In an implementation, solvent may be removed by lyophilization. In an implementation, the substantially pure saponin extract is characterized by negative ion mass spectrum indicating a most abundant species at mass-to-charge ratio m/z 1989 and impurities at m/z 1857, 1923, and 2480. In an implementation, the substantially pure saponin extract is characterized by a purity of at least 95% as determined by area-under-the-curve (AUC) of an analytical chromatography UV trace.

It is to be understood that one, some, or all of the properties of the various implementations described herein may be combined to form other implementations consistent with the present disclosure. These and other aspects of the present disclosure will become apparent upon reference to the following detailed description and attached drawings. All references disclosed herein are hereby incorporated by reference in their entirety as if each were incorporated individually.

### BRIEF DESCRIPTION OF THE DRAWINGS

FIG. 1 is a chromatogram showing UV_{210 nm} traces comparing traditional RP and polar RP stationary phase chemistries for the separation of a partially enriched Quillaja saponaria extract. VET-SAP® was separated on a traditional RP column, an analytical Gemini C18 (250×4.6 mm, 5 μm, 110 Å, dot dash line), and two analytical polar RP columns: Synergi Hydro RP (250×4.6 mm, 4 μm, 80 Å, dashed line) and Luna Omega Polar C18 (250×4.6 mm, 5 μm, 100 Å, solid line). Mobile phase A: Water (H₂O) with 0.1% formic acid and B: Acetonitrile (MeCN) with 0.1% formic acid, flow rate: 1 mL/min. Gradient condition: 0.0 min-20% B, 5.0 min-20% B, 25.0 min-80% B, 30.0 min-20% B. Fifty μL of VET-SAP® dissolved in H₂O at 1 mg/mL was injected. Peak containing QS-21 is indicated with an arrow.

FIG. 2 is a chromatogram showing UV_{210 nm} traces comparing mobile phase gradients on a polar RP column. VET-SAP® was separated on an analytical Luna Omega Polar C18 column (250×4.6 mm, 5 μm, 100 Å) with progressively narrowed gradients. Dot dash line trace gradient: 0.0 min-20% B, 5.0 min-20% B, 25.0 min-80% B, 30.0 min-20% B. Dashed line trace gradient: 0.0 min-30% B, 5.0 min-30% B, 25.0 min-70% B, 30.0 min-30% B. Solid trace gradient: 0.0 min-40% B, 5.0 min-40% B, 25.0 min-50% B, 27.5 min-80% B, 30.0 min-40% B. Fifty μL of VET-SAP® dissolved in H₂O at 1 mg/mL was injected. Mobile phase A: H₂O with 0.1% formic acid and B: MeCN with 0.1% formic acid, flow rate: 1 mL/min. Peak containing QS-21 is indicated with an arrow.

FIG. 3 is a chromatogram showing UV_{210 nm} traces of a partially enriched Quillaja saponaria extract separated by polar RP chromatography with progressively narrowed gradients. VET-SAP® dissolved in H₂O at 100 mg/mL was loaded onto a preparative Luna Omega Polar C18 column (250×50 mm, 5 μm, 100 Å). Mobile phase A: H₂O with 0.1% formic acid and B: MeCN with 0.1% formic acid. The flow rate was 85 mL/min. Solid line trace: 0.0 min-35% B, 15.0 min-35% B, 75.0 min-55% B, 80.0 min-55% B, 85.0 min-90% B, 100.0 min-90% B, 105.0 min-35% B, 125.0 min-35% B, 1 mL injection. dashed line trace: 0.0 min-37% B, 15.0 min-37% B, 60.0 min-50% B, 63.0 min-50% B, 68.0 min-90% B, 78.0 min-90% B, 83.0 min-37% B, 95.0 min-37% B, 3 mL injection. dotted line trace: 0.0 min-38% B, 15.0 min-38% B, 60.0 min-49% B, 62.0 min-49% B, 67.0 min-90% B, 72.0 min-90% B, 77.0 min-38% B, 85.0 min-38% B, 3 mL injection. Dot dash line trace: 0.0 min-40% B, 15.0 min-40% B, 40.0 min-47% B, 43.0 min-90% B, 46.0 min-90% B, 50.0 min-40% B, 60.0 min-40% B, 5.0 mL injection. Peak containing QS-21 is indicated with an arrow.

FIG. 4 is a chromatogram showing UV_{210 nm} traces comparing two different sample loading volumes of a partially enriched Quillaja saponaria extract. Five mL (solid line) and 10 mL (dashed) of 100 mg/mL VET-SAP® in H₂O was separated on preparative Luna Omega Polar C18 column (250×50 mm, 5 μm, 100 Å). Mobile phase A: H₂O with 0.1% formic acid and B: MeCN with 0.1% formic acid. The flow rate was 85 mL/min. Gradient condition: 0.0 min-40% B, 15.0 min-40% B, 40.0 min-47% B, 43.0 min-90% B, 46.0 min-90% B, 50.0 min-40% B, 60.0 min-40% B.

The main separation gradient with 10 mL sample loading was truncated at 45% B after elution of the QS-21 peak to further reduce run time and solvent use (dotted line). Gradient condition: 0.0 min-40% B, 15.0 min-40% B, 33.0 min-45% B, 36.0 min-90% B, 39.0 min-90% B, 43.0 min-40% B, 53.0 min-40% B. Other conditions are identical to those used for the dashed line trace. Dashed lines bracket the fractionation window of the dotted line trace collected as an intermediate purity saponin extract referred to as a “polar-RP intermediate purity saponin extract.” Peak containing QS-21 is indicated with an arrow.

FIG. 5 is a chromatogram showing a UV_{210 nm} trace of the separation of a partially enriched Quillaja saponaria extract by traditional RP chromatography. Three mL of 100 mg/mL VET-SAP® in water was separated on a preparative Gemini C18 column (250×21.2 mm, 5 μm, 110 Å). Gradient condition: 0.0 min-35% B, 11.5 min-35% B, 40.0 min-55% B, 42.5 min-55% B, 46.0 min-90% B, 48.0 min-35% B, 60.0 min-35% B, flow rate: 20 mL/min. Mobile phase A: H₂O with 0.1% formic acid and B: MeCN with 0.1% formic acid. Dotted lines bracket the fractionation window collected as an intermediate purity saponin extract referred to as a “traditional-RP intermediate purity saponin extract.” The fractionation window was identified by negative ion mode LC/MS analysis of individual fractions for singly charged QS-21 at m/z 1989 as the most abundant species above m/z 1200 among singly charged, intact saponins.

FIG. 6 is a chromatogram showing a UV_{210 nm} trace of the separation of a partially enriched Quillaja saponaria extract by polar RP chromatography. Ten mL of 100 mg/mL VET-SAP® in water was separated on a preparative Luna Omega Polar C18 column (250×50 mm, 5 μm, 100 Å). The preparative Luna Omega Polar C18 column was fitted with a 15×30.0 mm guard column. Mobile phase A: H₂O with 0.1% formic acid and B: MeCN with 0.1% formic acid. Gradient condition: 0.0 min-40% B, 15.0 min-40% B, 40.0 min-47% B, 43.0 min-90% B, 46.0 min-90% B, 50.0 min-40% B, 60.0 min-40% B, flow rate: 85 mL/min. Dashed lines bracket the fractionation window collected as an intermediate purity saponin extract referred to as a “polar-RP intermediate purity saponin extract.”

FIG. 7 shows a negative ion LC/Q-TOF-MS spectrum of the intermediate purity saponin extract collected from the indicated fractionation window of FIG. 5. Singly deprotonated QS-21 is indicated by the peak at m/z 1,989.

FIG. 8 shows a negative ion LC/Q-TOF-MS spectrum of the intermediate purity saponin extract collected from the indicated fractionation window of FIG. 6. Singly deprotonated QS-21 is indicated by the peak at m/z 1,989.

FIG. 9 is a chromatogram showing a UV_{210 nm} trace of a traditional-RP intermediate purity saponin extract separated on a second, different traditional RP chromatography column. The source of this traditional-RP intermediate purity saponin extract is indicated by the bracket fraction shown in FIG. 5. The traditional-RP intermediate purity saponin extract was lyophilized and the lyophilized powder was dissolved in 45:55 MeCN:H₂O, 0.1% formic acid at 20 mg/mL. Two and a half mL of the resuspended intermediate purity saponin extract was separated on a preparative Polaris C8 column (250×21.2 mm, 5 um, 110 Å). Mobile phase A: H₂O with 0.1% formic acid, mobile phase B: MeCN with 0.1% formic acid. Gradient condition: 0.0 min-35% B, 9.0 min-35% B, 39.0 min-60% B, 43.0 min-60% B, 50.0 min-90% B, 57.0 min-35% B, 60.0 min-35% B, flow rate: 20 mL/min. Dashed lines bracket the fractionation window collected as a purified sample referred to as “dual RP chromatography purified QS-21.”

FIG. 10 is a chromatogram showing UV_{210 nm} traces comparing separation of a traditional-RP intermediate purity saponin extract (solid line) and dual RP chromatography purified QS-21 (dashed line) on an unbonded silica column. An analytical Kinetex HILIC column (250×4.6 mm, 5 μm, 100 Å) was run isocratically in 80:20 MeCN:H₂O, 5 mM ammonium acetate (pH 5.8) at 1 mL/min flow rate. Twenty μL of each sample dissolved at 1 mg/mL in 70:30 MeCN:H₂O, 5 mM ammonium acetate (pH 5.8) was injected.

FIG. 11 is a chromatogram showing UV_{210 nm} traces comparing the effects of two buffer types with different pH on the separation of intermediate purity saponin extract on an analytical Kinetex HILIC column (250×4.6 mm, 5 μm, 100 Å). Column was run isocratically with 80:20 MeCN:H₂O and either 5 mM ammonium acetate at pH 5.8 (solid line) or 5 mM ammonium formate at pH 3.2 (dashed line) at 1 mL/min flow rate. Twenty μL of traditional-RP intermediate purity saponin extract dissolved at 1 mg/mL in 70:30 MeCN:H₂O buffered by corresponding salt in the mobile phase was injected.

FIG. 12 is a chromatogram showing UV_{210 nm} traces of traditional-RP intermediate purity saponin extract (solid line) and dual RP chromatography purified QS-21 (dashed line) separated on an amide-functionalized HILIC column. An analytical TSKgel-Amide 80 column (250×4.6 mm, 5 μm, 100 Å) was run isocratically in 80:20 MeCN:H₂O, 5 mM ammonium acetate (pH 5.8) at 1 mL/min flow rate. Twenty μL of each sample dissolved in 70:30 MeCN:H₂O, 5 mM ammonium acetate (pH 5.8) at 1 mg/mL was injected.

FIG. 13 is a chromatogram showing UV_{210 nm} traces of traditional-RP intermediate purity saponin extract (solid line) and dual RP chromatography purified QS-21 (dashed line) separated on an amino-functionalized HILIC column. An analytical Zorbax NH₂ column (250×4.6 mm, 5 μm, 70 Å) was run isocratically in 60:40 MeCN:H₂O, 5 mM ammonium acetate (pH 5.8) at 1 mL/min flow rate. Twenty μL of each sample dissolved in the mobile phase at 1 mg/mL was injected.

FIG. 14 is a chromatogram showing UV_{210 nm} traces comparing three different mobile phase conditions on the separation of intermediate purity saponin extract on an analytical Zorbax NH₂ column (250×4.6 mm, 5 μm, 70 Å). Column was run isocratically in 50:50 (solid line), 60:40 (dashed line) or 65:35 (dot dash line) MeCN:H₂O, 5 mM ammonium acetate (pH 5.8) at 1 mL/min flow rate. Twenty μL of traditional-RP intermediate purity saponin extract dissolved in the corresponding mobile phases at 1 mg/mL was injected.

FIG. 15 is a chromatogram showing a UV_{210 nm} trace of intermediate purity saponin extract separated on an amino-functionalized HILIC column. An analytical Luna NH₂ column (250×4.6 mm, 5 μm, 100 Å) was run isocratically in 50:50 MeCN:H₂O, 5 mM ammonium acetate (pH 5.8) at 1 mL/min flow rate. Twenty μL of traditional-RP intermediate purity saponin extract dissolved in the mobile phase at 1 mg/mL was injected.

FIG. 16 is a chromatogram showing UV_{210 nm} traces of traditional-RP intermediate purity saponin extract (solid line) and dual RP chromatography purified QS-21 (dashed line) separated on a sulfobetaine-functionalized zwitterionic HILIC column. An analytical Syncronis HILIC column (250×4.6 mm, 5 μm, 100 Å) was run isocratically in 80:20 MeCN:H₂O, 5 mM ammonium formate (pH 3.2). Twenty μL of each sample dissolved in 70:30 MeCN:H₂O, buffered by corresponding salts in the mobile phase at 1 mg/mL was injected.

FIG. 17 is a chromatogram showing UV_{210 nm} traces of traditional-RP intermediate purity saponin extract (solid line) and dual RP chromatography purified QS-21 (dashed line) separated on an analytical Syncronis HILIC column (250×4.6 mm, 5 μm, 100 Å). Column was run isocratically in 80:20 MeCN:H₂O, 5 mM ammonium acetate (pH 5.8) at 1 mL/min flow rate. Twenty μL of each sample dissolved in 70:30 MeCN:H₂O, buffered by corresponding salts in the mobile phase at 1 mg/mL was injected.

FIG. 18 is a chromatogram showing UV_{210 nm} traces of the separation of intermediate purity saponin extract provided by two different amounts of organic solvent in the mobile phase run on a hybrid amide/amino-functionalized HILIC column. Traditional-RP intermediate purity saponin extract was separated on an analytical Luna Omega SUGAR column (250×4.6 mm, 3 μm, 100 Å). Twenty μL of traditional-RP intermediate purity saponin extract dissolved in 70:30 MeCN:H₂O, 5 mM ammonium acetate (pH 5.8) at 1 mg/mL was injected. Column was run isocratically in 70:30 (solid line) or 75:25 (dashed line) MeCN:H₂O, 5 mM ammonium acetate (pH 5.8) at 1 mL/min flow rate.

FIG. 19 is a chromatogram showing UV_{210 nm} traces comparing loading volumes on a first zwitterionic HILIC column. Traditional-RP intermediate purity saponin extract was separated on an analytical Syncronis HILIC column (250×4.6 mm, 5 μm, 100 Å). Two μL (solid line), 5 μL (dashed line), 30 μL (dot dash line) and 60 μL (dotted line) of traditional-RP intermediate purity saponin extract dissolved in 65:35 MeCN:H₂O, 5 mM ammonium acetate (pH 5.8) at 40 mg/mL were injected. Column was run isocratically in 80:20 MeCN:H₂O, 5 mM ammonium acetate (pH 5.8) at 1 mL/min flow rate.

FIG. 20 is a chromatogram showing UV_{210 nm} traces comparing loading volumes on a second zwitterionic HILIC column. Traditional-RP intermediate purity saponin extract was separated on an analytical Sequant ZIC-HILIC column (250×4.6 mm, 5 μm, 200 Å). Five μL (dashed line) and 30 μL (solid line) of traditional-RP intermediate purity saponin extract dissolved in 65:35 MeCN:H₂O, 5 mM ammonium acetate (pH 5.8) at 40 mg/mL were injected. Column was run isocratically in 80:20 MeCN:H₂O, 5 mM ammonium acetate (pH 5.8) at 1 mL/min flow rate.

FIG. 21 is a chromatogram showing UV_{210 nm} traces comparing loading volumes on a preparative version of the zwitterionic HILIC column used in FIG. 16, FIG. 17, and FIG. 19. Traditional-RP intermediate purity saponin extract was separated on a preparative Syncronis HILIC column (21.2×250 mm, 5 μm, 100 Å with a 10 mm length guard column) with two sample loading volumes of 250 μL (dashed line) and 600 μL (solid line). The respective amounts of each sample dissolved in 65:35 MeCN:H₂O, 5 mM ammonium acetate (pH 5.8) at 40 mg/mL were injected. Column was run isocratically in 80:20 MeCN:H₂O, 5 mM ammonium acetate (pH 5.8) at 20 mL/min flow rate.

FIG. 22 is a chromatogram showing UV_{210 nm} traces comparing loading volumes of polar-RP intermediate purity saponin extract on the zwitterionic HILIC column of FIG. 16. The samples were separated on a preparative Syncronis HILIC column (21.2×250 mm, 5 μm, 100 Å with a 10 mm guard column) with the sample loading volumes of 250 μL (dashed line) and 600 μL (solid line). The polar-RP intermediate purity saponin extract dissolved in 65:35 MeCN:H₂O, 5 mM ammonium acetate (pH 5.8) at 40 mg/mL was injected. Column was run isocratically in 80:20 MeCN:H₂O, 5 mM ammonium acetate (pH 5.8) at 20 mL/min flow rate. Dotted lines bracket the fraction collection window on the 600 μL (solid) loading trace. This fraction is referred to as “substantially pure QS-21.”

FIG. 23 is a chromatogram showing a UV_{210 nm} trace of traditional-RP intermediate purity saponin extract separated on the zwitterionic HILIC column of FIG. 16. Fractions 1, 2, and 3 indicated as bracketed by dotted lines were separately collected. The column is a preparative Syncronis HILIC column (21.2×250 mm, 5 μm, 100 Å). Six-hundred pi sample dissolved in 65:35 MeCN:H₂O, 5 mM ammonium acetate (pH 5.8) at 40 mg/mL was injected. Column was run isocratically with 80:20 MeCN:H₂O, 5 mM ammonium acetate (pH 5.8) at 20 mL/min flow rate.

FIG. 24 is a mass spectrum generated by Liquid Chromatography/Triple Quadrupole-Mass Spectrometry (LC/QqQ-MS) of fraction 1 from FIG. 23.

FIG. 25 is a mass spectrum generated by LC/QqQ-MS of fraction 2 from FIG. 23.

FIG. 26 is a mass spectrum generated by LC/QqQ-MS of fraction 3 from FIG. 23.

FIG. 27 is a chromatogram showing a UV_{210 nm} trace of a substantially pure saponin extract containing QS-21 characterized on an analytical Vydac C4 column (250×4.6 mm, 5 μm, 300 Å). Mobile phase A: H₂O with 0.1% formic acid, mobile phase B: MeCN with 0.1% formic acid, flow rate: 1 mL/min. The substantially pure QS-21 indicated by the fraction shown in FIG. 22 was lyophilized. Fifty μL of the lyophilized product was dissolved in 35% B at 1 mg/mL was injected. Gradient condition: 0.0 min-35% B, 20.0 min-40% B, 21.0 min-40% B, 26.0 min-90% B, 34.0 min-90% B, 35.0 min-35% B. The trace shows a minor peak at about 15.5 minutes and a major peak about 18.0 minutes.

FIG. 28 is a mass spectrum generated by LC/Q-TOF-MS of substantially pure QS-21 in negative ion mode. The substantially pure QS-21 was obtained from the fraction indicated in FIG. 22. The most abundant ion is singly charged QS-21 with an m/z of 1989.

FIG. 29 is a negative ion mass spectrum generated by LC/Quadrupole Time-of-Flight (Q-TOF) MS of the minor peak shown in FIG. 27. Note that the signal intensity is one order of magnitude lower than in FIG. 28, thus low m/z background ions appear more prominent.

FIG. 30 is a negative ion mass spectrum generated by LC/Q-TOF MS of the major peak shown in FIG. 27.

### DETAILED DESCRIPTION

The present disclosure is generally directed to a new orthogonal chromatographic process for the purification of saponins from Quillaja saponaria. This disclosure is also directed to saponin extracts containing QS-21 with greater than 95% purity and characterized by negative ion mass spectrum indicating a most abundant species with a mass-to-charge ratio (m/z) of 1989 and significant impurities only at m/z 1857, 1923, and 2480.

The chromatographic process of this disclosure provides a significant and unexpected improvement over other techniques for purifying saponins from Quillaja saponaria. This chromatographic process comprises two orthogonal chromatographic steps in series, a reversed-phase (RP) chromatographic step on a polar RP column followed by a HILIC chromatographic step. The inventors of this disclosure are not aware of these two chromatography steps being previously combined in this order for the purpose of preparative purification of Quillaja saponaria saponins. Without being bound by theory, it is believed that due to the amphiphilic nature of Quillaja saponaria saponins, the retention and selectivity provided by this combination of orthogonal chromatographic steps achieve higher yield and purity than other methods. Additionally, this method can purify partially enriched Quillaja saponaria extract from multiple sources or extraction techniques without any strict requirement on the composition of the extract. Commercially available Quillaja saponaria extracts (e.g. VET-SAP® and Quil-A®) may be purified by this method without any pre-treatment. Moreover, due to its relative simplicity, this novel technique can be easily adapted for large-scale production.

Multiple techniques could be used to purify saponins from Quillaja saponaria. These techniques include, but are not limited to, solvent extraction, adsorption, ultrafiltration, liquid chromatography, counter-current chromatography, crystallization, and supercritical fluid extraction. These techniques may be combined in many different orders and there are multiple variations possible for each technique. The difficulty and challenge of developing effective and selective methods for the extraction and isolation of bioactive natural products is well recognized and discussed in Zhang, Q., Lin, L. & Ye, W. Techniques for extraction and isolation of natural products: a comprehensive review. Chin Med 13, 20 (2018). Saponins are known to be particularly difficult to separate because they occur naturally as a mixture of structurally similar compounds of similar polarity. (Majinda RR. Extraction and isolation of saponins. 864 Methods Mol Biol. 415-26. (2012) It is not possible to accurately predict in advance which combination of techniques will prove effective for purifying saponin extracts.

Even if only chromatographic techniques are considered, there is still a large number of possible techniques that may be used for purifying saponins. General classes of chromatographic techniques include, but are not limited to, size exclusion chromatography (SEC), ion exchange (IEX) chromatography, affinity chromatography, normal-phase (NP) chromatography, reversed-phase (RP) chromatography, hydrophobic interaction chromatography (HIC), and hydrophilic interaction chromatography (HILIC). These chromatographic techniques are known to those of ordinary skill in the art and are explained in greater detail below. Each of these general classes of chromatographic techniques may also include many variations and subclasses of techniques. Any two, or more, chromatographic techniques may be combined in any order generating an even larger number of potential purification techniques. It is difficult to predict the effectiveness for any combination of chromatographic techniques when applied to purification of a complex mixture of similar molecules such as saponins. In advance of testing, the skilled artisan may be able to identify some techniques as unsuitable but would have no way to predict which set of chromatographic techniques would provide superior separation and purification.

Any technique not specifically described herein is performed according to standard protocols understood by one of ordinary skill in the art. Examples of the knowledge of one of ordinary skill in the art with respect to chromatography is provided by the Handbook of HPLC, 2^{nd} ed., edited by Danilo Corradini, Taylor & Francis Group (2010) and Liquid Chromatography: Fundamentals and Instrumentation, edited by Salvatore Fanali, et al., Elsevier (2013).

### A. Definitions

The following terms have the following meanings unless otherwise indicated. Any undefined terms have their art-recognized meanings. Art recognized meanings may be determined by reference to any of the documents described in this disclosure as well as to other publications. The IUPAC. Compendium of Chemical Terminology, 2^{nd} ed. (the “Gold Book”) compiled by A. D. McNaught and A. Wilkinson, Blackwell Scientific Publications, Oxford (1997) provides definitions for chromatography terms.

As used herein and in the appended claims, the singular forms “a,” “an” and “the” include plural reference unless the context clearly dictates otherwise.

It is understood that aspects and implementations of the disclosure described herein include “comprising,” “consisting,” and “consisting essentially of” aspects and implementations.

In the present description, the terms “about,” “approximately,” similar referents and “consisting essentially of” mean+/−10% of the indicated range, value, or structure, unless otherwise indicated.

The use of the alternative (e.g., “or”) should be understood to mean either one, both, or any combination thereof of the alternatives.

The terms “in part,” “at least in part,” “a portion,” or similar referents are to be construed as meaning at least a portion or part of the whole including up to the entire noun referenced.

As used herein, the terms “include,” “have” and “comprise” are used synonymously, which terms and variants thereof are intended to be construed as non-limiting.

The term “isolated” means a molecule has been removed from its natural environment.

“Purified” means that the molecule has been increased in purity, such that it exists in a form that is more pure than it exists in its natural environment and/or when initially synthesized and/or amplified under laboratory conditions. Purity is a relative term and does not necessarily mean absolute purity.

The term “dried” means that substantially all solvent has been removed. A dried saponin extract will typically contain less than 5% solvent w/w (such as less than 5% water w/w). A dried saponin extract will typically contain 100 ppm or less organic solvent (w/w).

The term “orthogonal chromatography” means the combination of two or more chromatographic techniques that differ significantly in chromatographic selectivity and mechanism of action.

The term “saponin” means a class of glycosides having one or more hydrophilic glycone moieties combined with a lipophilic triterpene or steroid derivative. Saponins are noted for forming colloidal solutions in water which foam on shaking, and for precipitating cholesterol.

The term “partially enriched Quillaja saponaria extract” means a Quillaja saponaria bark extract that contains a complex mixture of polyphenolics and a number of different saponins. Saponin concentration and purity are increased relative to the concentration and purity of the saponin in Quillaja saponaria bark. A partially enriched Quillaja saponaria extract may be a solution or a dried powder. A partially enriched Quillaja saponaria extract may be referred to as a crude saponin extract.

The term “intermediate purity saponin extract” means a partially enriched Quillaja saponaria extract that has been purified by a single chromatographic technique to remove impurities and optionally also to remove one or more other types of saponins. A partially enriched Quillaja saponaria extract may include more than one type of saponin. The term intermediate purity saponin extract includes both traditional-RP intermediate purity saponin extracts and polar-RP intermediate purity saponin extracts. An intermediate purity saponin extract may also be referred to as a semi-pure saponin extract.

The term “traditional-RP intermediate purity saponin extract” means an intermediate purity saponin extract in which the single chromatographic technique used to purify partially enriched Quillaja saponaria extract is RP chromatography performed on a traditional RP column.

The term “polar RP intermediate purity saponin extract” means an intermediate purity saponin extract in which the single chromatographic technique used to purify partially enriched Quillaja saponaria extract is RP chromatography performed on a polar RP column.

The term “dual RP chromatography purified QS-21” means a traditional-RP intermediate purity saponin extract that contains QS-21 further purified by separation with a second traditional RP chromatography step. For example, dual RP chromatography purified QS-21 may be purified by separation on a traditional RP C18 column followed by separation on a traditional RP C8 column. Dual RP chromatography purified QS-21 is a type of purified QS-21.

The term “substantially pure saponin extract” means an extract that predominately contains a single saponin of interest and is substantially free from impurities and other saponins normally associated with the saponin of interest in its natural state. A substantially pure saponin extract exhibits constant and reproducible chromatographic response, elution profiles, and biologic activity. Substantially pure saponin extracts may include mixtures of regioisomers or compositional isomers of the saponin of interest. Regioisomers are a class of constitutional isomers that have the same functional groups but attached at different positions. Compositional isomers specifically in the context of saponins are isomeric saponins that share the same molecular weight but are differentially functionalized with one or more terminal sugars. A substantially pure saponin extract may include solvents such as a chromatographic mobile phase.

The term “substantially pure QS-21” means a substantially pure saponin extract in which the saponin of interest is QS-21.

The term “shoulder peak” means a smaller peak adjacent to a larger peak on a chromatogram that is not baseline separated from the larger peak.

An “individual” or a “subject” is any mammal. Mammals include, but are not limited to humans, primates, farm animals, sport animals, pets (such as cats, dogs, horses), and rodents.

### B. Partially Enriched Quillaja saponaria Extracts

Purification of saponins ultimately begins with the bark of Quillaja saponaria.

The bark may be processed by any of multiple known techniques to create a partially enriched Quillaja saponaria extract that contains a complex mixture of polyphenolics and a number of different saponins. One illustrative technique for producing a partially enriched Quillaja saponaria extract is provided by Example 1 of U.S. Pat. No. 5,057,540. This technique uses dialysis and methanol extraction to process the bark of Quillaja saponaria Molina. Other procedures for producing partially enriched Quillaja saponaria extract are provided in Dalsgaard, K., Saponin adjuvants III. Isolation of a substance from Quillaja saponaria Molina with adjuvant activity in foot-and-mouth disease vaccines, in Archiv fur die gesamte virusforschung. 1974, Springer-Verlag. p. 243-254 and Kensil, C. A. and D. J. Marciani, Saponin Adjuvant. 1991, Cambridge Biotech Corporations, Worcester, Mass.:

United States.

The commercially available products VET-SAP® and Quil-A® are examples of partially enriched Quillaja saponaria extracts. VET-SAP® and Quil-A® are both supplied as lyophilized, dry powders. VET-SAP® is available from Desert King International (San Diego, Calif., USA) and Quil-A® is available from several sources including Superfos (Vedbaek, Denmark) and Croda International Plc (East Yorkshire, United Kingdom). VET-SAP® is described in WO 2017/09133. Preparation of Quil-A® is described in Dalsgaard, Archiv fuer die gesamte Virus-forschung 44:243 (1974). Characterization of Quil-A® by HPLC is shown in U.S. Pat. No. 5,057,540. The exact content of individual saponins in commercially available extracts are not specified by the manufacturers and have batch-to-batch variation.

### C. Chromatographic Techniques

Size exclusion chromatography (SEC) is a technique in which samples are separated according to their hydrodynamic sizes. The molecules in a sample travel through the pores of the stationary phase; smaller molecules have longer elution times as they have access to larger pore volumes and vice versa. The mobile phase can be either organic or aqueous depending on the application need. SEC is commonly used for the separation of synthetic polymers and biomolecules.

Ion exchange (IEX) chromatography is a technique in which charged molecules are separated as a result of ionic interaction with oppositely charged functional groups on the stationary phase in exchange with ions of the same charge in the mobile phase. Sample retention or elution is modulated by the pH and/or ion strength of the mobile phase. There are two IEX modes, cation exchange, where a negatively charged stationary surface is used to retain and separate positively charged samples, and anion exchange, where a positively charged stationary surface is used to retain and separate negatively charged samples. IEX chromatography is most commonly used to separate biological molecules such as proteins, peptides, amino acids, and nucleotides which contain ionizable groups.

Another technique that is frequently used for purifying biological samples is affinity chromatography. Affinity chromatography isolates a molecule by exploiting its highly specific interaction with a functionalized stationary phase. Examples of such interaction include those between antigen and antibody, enzyme and substrate, receptor and ligand, and metal and chelator. This affinity can be the result of intrinsic properties of the target molecule or can be imparted by the addition of a purification tag. Typically, the sample is loaded onto the column to allow binding of the target molecule to the stationary surface. A washing buffer is then used to remove non-target molecules that are unbound or weakly bound to the stationary phase through non-specific interactions. An elution buffer, which disrupts the specific interaction between the target molecule and the stationary surface, is finally introduced to elute the molecule of interest.

Normal-phase (NP) chromatography separates samples based on their interaction with a polar stationary surface. Examples of polar interactions include hydrogen bonding and dipole-dipole interaction. The higher the polarity of a molecule, the stronger it is adsorbed to the stationary phase and the longer it takes to elute. Mobile phase typically starts at a low polarity to allow for sample adsorption to the stationary phase and increases in polarity over time to elute samples in order of their adsorption strengths. Classic NP chromatography uses strictly organic solvents as the mobile phase and is thus applicable to organic-soluble compounds.

Reversed-phase (RP) chromatography, as the name suggests, is the opposite of NP chromatography. Sample molecules are retained and separated based on their interaction with a non-polar stationary surface, such as one that is functionalized with hydrocarbon groups. The lower the polarity of a molecule, the stronger it is retained by the stationary phase and the longer it takes to elute. Mobile phase typically consists of a blend of water with a miscible, polar organic solvent, Molecules are eluted in order of decreasing polarity with increasing organic content in the mobile phase. Given its robustness and versatility, RP chromatography is the most commonly used chromatography technique with applications ranging from small molecules to peptides, proteins, and oligonucleotides. Traditional RP stationary phases typically consist of linear or aromatic hydrocarbons bonded on silica with remaining surface silanols covered or end-capped.

Polar RP columns are a recent variation of RP stationary phase chemistry used in RP chromatography. Polar reversed-phase columns contain both polar and non-polar functional groups on the stationary surface. This type of solid phase offers retention and selectivity toward both polar and non-polar moieties on samples. To emphasize the difference between RP chromatography run on a polar RP column and a traditional RP column, the latter may be referred to as traditional RP chromatography.

Hydrophobic interaction chromatography (HIC) may be viewed as a variant of RP chromatography in that samples are separated based on their hydrophobicity. It is a technique used to separate biomolecules such as proteins, where operating in less denaturing conditions is desirable. The sample is loaded onto the column in a high-salt aqueous buffer, which reduces its solubility and encourages its binding to a moderately hydrophobic stationary phase through hydrophobic interaction. Decreasing the salt content in the mobile phase then elutes molecules in order of increasing hydrophobicity. Sample elution may also be assisted by mild organic modifiers or detergents.

Hydrophilic interaction chromatography (HILIC) is a chromatographic technique that shares some similarities with several of the above-mentioned techniques, but its separation mechanism is distinct from those of the others. HILIC uses normal-phase type polar stationary phases and reversed-phase type mobile phases. Typical HILIC mobile phase consists of a mixture of a water-miscible aprotic solvent such as acetonitrile and a small amount of water. The current, conventional understanding of this technique is that a stagnant water layer is formed on the surface of the polar stationary phase, creating a liquid/liquid interface with the organic-rich mobile phase. Sample molecules partition between these two layers in response to changes in the mobile phase polarity. In addition, samples may also participate in dipole-dipole interaction, hydrogen bonding, or ionic interaction with the stationary phase depending on the exact chemistry involved. Buffer salts may be added to control the mobile phase pH and ion strength. HILIC finds applications in the separation of a variety of polar compounds, particularly with glycosylated molecules such as glycosides and glycoproteins.

### D. First Chromatography Purification Step

The first chromatography step is performed by reversed-phase chromatography that may be implemented as traditional RP chromatography or polar RP chromatography.

In an implementation, the first chromatography purification step provided in this disclosure uses traditional RP chromatography to purify a partially enriched Quillaja saponaria extract. The first chromatographic purification step produces an intermediate purity saponin extract that may be referred to as a traditional-RP intermediate purity saponin extract.

In an implementation, the first chromatography purification step provided in this disclosure uses polar RP chromatography to purify a partially enriched Quillaja saponaria extract. The first chromatographic purification step produces an intermediate purity saponin extract that may be referred to as a polar-RP intermediate purity saponin extract. Without being bound by theory, it is believed that due to the amphiphilic nature of saponins, RP chromatography run on a polar RP column provides better retention and selectivity toward both the non-polar and polar moieties on saponins than other chromatographic techniques such as traditional RP chromatography. However, the extent of any improvement over traditional RP chromatography, or any other chromatographic technique, was not known and was not reasonably predictable prior to testing.

In an implementation, the partially enriched Quillaja saponaria extract is a powder. The powder may be referred to as a dried, partially enriched Quillaja saponaria extract. The powder may be dissolved in water (e.g. ultrapure water) at a concentration of about 1-100 mg/mL. In illustrative implementations, the powder is dissolved at a concentration of 1, 2, 3, 4, 5, 6, 7, 8, 9, 10, 15, 20, 25, 30, 35, 40, 45, 50, 55, 60, 65, 70, 75, 80, 85, 90, 95, or 100 mg/mL.

In an implementation for analytical RP chromatography, about 30-70 μg of dried, partially enriched Quillaja saponaria extract dissolved in water is separated on an analytical RP column. In illustrative implementations, the amount of dried, partially enriched Quillaja saponaria extract is 30, 35, 40, 45, 50, 55, 60, 65, or 70 μg. In an implementation, about 30-70 μL of partially enriched Quillaja saponaria extract dissolved in water is injected into an analytical polar RP column. In illustrative implementations, 30, 35, 40, 45, 50, 55, 60, 65, or 70 μL is injected. In an implementation, partially enriched Quillaja saponaria extract is dissolved in water at 1 mg/mL and 50 μL (50 μg) is injected into an analytical polar RP column.

In an implementation for preparative RP chromatography, about 80-1800 mg of dried, partially enriched Quillaja saponaria extract dissolved in water is separated on a preparative polar RP column. In illustrative implementations, 80, 90, 100, 150, 200, 250, 300, 350, 400, 450, 500, 550, 600, 650, 700, 750, 800, 850, 900, 950, 1000, 1050, 1100, 1150, 1200, 1200 1,300, 1350, 1400, 1450, 1500, 1550, 1600, 1650, 1700, 1750, or 1800 mg of partially enriched Quillaja saponaria extract is dissolved. In an implementation, partially enriched Quillaja saponaria extract is dissolved in water at a concentration of about 80-120 mg/mL and about 1-15 mL is injected into a preparative polar RP column. In illustrative implementations, the concentration is 80, 85, 90, 95, 100, 105, 110, 115, or 120 mg/mL. In illustrative implementations, 1, 2, 3, 4, 5, 6, 7, 8, 9, 10, 11, 12, 13, 14 or 15 mL is injected. In an implementation, partially enriched Quillaja saponaria extract is dissolved in water at 100 mg/mL and 3 mL (300 mg) is injected into a preparative traditional RP chromatography column. In an implementation, partially enriched Quillaja saponaria extract is dissolved in water at 100 mg/mL and 10 mL (1000 mg) is injected into a preparative polar RP chromatography column. The amount of a saponin, such as QS-21, in the partially enriched Quillaja saponaria extract will vary based on the amount of the saponin in the bark and the specific technique used for the extraction.

The partially enriched Quillaja saponaria extract dissolved in water may be filtered prior to separation by chromatography. In an implementation, the dissolved sample may be filtered on a 0.2 μm filter. One suitable filter that may be used is a disposable 0.2 polyethersulfone member filter unit (Corning Inc., Corning, NJ, USA). Following filtration, the partially enriched Quillaja saponaria extract dissolved in water may be injected into a chromatography column at any of the volumes listed above or described in the examples.

In an implementation, the traditional RP or the polar RP solid phase may comprise non-polar linear alkyl or aryl groups chemically bonded to porous or non-porous silica, ceramic, or polymeric microparticles or monoliths. The aryl groups may be further functionalized as in pentafluorophenyl. In an implementation, the linear alkyl group is octadecyl (C18), octyl(C8), butyl (C4), or triacontyl (C30). In an implementation of polar RP chromatography, polar moieties may be incorporated by end-capping the free silanols or embedding within the non-polar groups to provide retention of both non-polar as well as polar compounds via polar interactions, hydrogen bonding, or electrostatic interactions. In an implementation, the solid phase may have a particle size of 1.3, 1.6, 1.7, 1.8, 2.5, 2.6, 2.7, 3, 3.5, 3.6, 4, 5, 6, 7, 7.5, 8, 9, or 10 μm. In an implementation, particles in the solid phase may have a pore size of 60, 70, 80, 90, 100, 110, 120, 130, 140, 150, 160, 170, 180, 190, 200, 210, 220, 230, 240, 250, 260, 270, 280, 290, or 300 Å. For example, traditional RP chromatography may be performed on a Gemini C18 column. For example, polar RP chromatography may be performed on a Luna® Omega Polar C18 column or a Synergi™ Hydro RP column.

In an implementation, the mobile phase comprises water and an organic solvent. The organic solvent may be acetonitrile, methanol, tetrahydrofuran, ethanol, or isopropanol. In an implementation, the mobile phase comprises a volatile modifier. The volatile modifier may be formic acid, trifluoroacetic acid (TFA), or acetic acid. In an implementation, the mobile phase may comprise 20-80% organic solvent.

The polar RP chromatography may be performed under isocratic conditions or under a solvent gradient (e.g., continuous or stepped). In an implementation, the solvent gradient may range from 20-80%, 30-70%, 40-60%, 40-55%, 35-55%, 37-50%, 38-49%, or 40-47% organic solvent.

In an implementation, the runtime of the polar RIP chromatography step until elution of the peak containing QS-21 may be less than 20, 19, 18, 17, 16, or 15 minutes on an analytical polar RP column at a flow rate of 1 mL/min. In an implementation, the runtime of the polar RP chromatography step until elution of the peak containing QS-21 may be less than 45, 44, 43, 42, 41, 40, 39, 38, 37, 36, 35, 34, 33, 32, 31, 30, 29, 28, or 27 minutes on a preparative polar RP column at a flow rate of 85 mL/min.

In an implementation, fractions of eluate corresponding to one or more peaks in the chromatogram generated by polar RP chromatography may be collected and analyzed for saponin content. In an implementation, the saponin content may be analyzed by LC-MS. Fractions corresponding to a peak containing the saponin of interest may be collected. In an implementation, only fractions corresponding to the peak itself are collected. Fractions corresponding to left and right shoulder peaks that are not baseline separated from the main peak of interest may be excluded. The saponin of interest may be QS-7, QS-17, QS-18, or QS-21.

The intermediate purity saponin extract collected from polar RP chromatography containing the saponin of interest (e.g. QS-7, QS-17, QS-18, or QS-21) is the input for the second chromatography purification step. In an implementation, the fractions collected from polar RP chromatography may be separated in a second chromatography purification step without drying or removal of the mobile phase. The fractions may be filtered prior to additional chromatographic separation. In an implementation, the filter may comprise a 0.2 μm polytetrafluoroethylene (PTFE) membrane.

In an implementation, the intermediate purity saponin extract may be dried and lyophilized. Drying may be performed by evaporating the organic solvent wider a stream of nitrogen or filtered air. The dried product may then be lyophilized to remove the water content. The dried product may be stored frozen at, for example, −20 or −80° C.

### E. Second Chromatography Purification Step

In an implementation, the second chromatography purification step uses HILIC chromatography to further purify the intermediate purity saponin extract obtained from the first step and obtain a substantially pure saponin extract. Without being bound by theory, it is believed that the orthogonality between polar RP chromatography and HILIC chromatography provides enhanced separation leading to a final purified product with greater purity than other combinations of chromatographic techniques. Yield and purity are also better than purification using two traditional RP chromatograph steps.

The intermediate purity saponin extract, if available as a dried powder, may be dissolved in a mixture of organic solvent and water. In an implementation, the mixture of organic solvent and water may be the same as the mobile phase that will be used for HILIC chromatography. In various implementations, the intermediate purity saponin extract may be dissolved in acetonitrile and water combined at a ratio of 45:55, 50:50, 60:40, 65:35, or 70:30, (v:v). In implementations, the intermediate purity saponin extract may be dissolved in a mobile phase that comprises about 0.1-0.3% formic acid, about 3-7 mM ammonium acetate, or about 3-7 mM ammonium formate. In implementations, the intermediate purity saponin extract may be dissolved in a mobile phase at pH of about 3-8, about 3-6, or about 5.8.

In an implementation for analytical HILIC chromatography, about 2-100 μg of dried, intermediate purity saponin extract dissolved in a mixture of organic solvent and water is separated on an analytical HILIC column. In illustrative implementations, the amount of dried, intermediate purity saponin extract is 2, 3, 4, 5, 6, 7, 8, 9, 10, 15, 20, 25, 30, 35, 40, 45, 50, 55, 60, 65, 70, 75, 80, 85, 90, 95, or 100 jig. In an implementation, the intermediate purity saponin extract dissolved in a mixture of organic solvent water at a concentration of about 1, 2, 3, 4, or 5 mg/mL is injected into an analytical HILIC column. In an implementation, about 2-60, 5-40, 10-30, or 20 μL of intermediate purity saponin extract dissolved in a mixture of organic solvent and water is injected into an analytical HILIC column. In illustrative implementations, 2, 3, 4, 5, 6, 7, 8, 9, 10, 15, 20, 25, 30, 35, 40, 45, 50, 25, or 60 μL is injected. In an implementation, intermediate purity saponin extract is dissolved in a mixture of organic solvent in water at 1 mg/mL and 20 μL (20 μg) is injected into an analytical HILIC column.

In an implementation for preparative HILIC chromatography, about 4-28 mg of dried, intermediate purity saponin extract dissolved in a mixture of organic solvent and water is separated on a preparative HILIC column. In illustrative implementations, the amount of dried, intermediate purity saponin extract is 4, 5, 6, 7, 8, 9, 10, 11, 12, 13, 14, 15, 16, 17, 18, 19, 20, 21, 22, 23, 24, 25, 26, 27, or 28 mg. In an implementation, intermediate purity saponin extract is dissolved in a mixture of organic solvent and water at about 20-60 mg/mL and about 200-700 μL is injected into a preparative HILIC column. In illustrative implementations, the intermediate purity saponin extract is dissolved in a mixture of organic solvent and water at a concentration of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg/mL. In illustrative implementations, 200, 250, 300, 350, 400, 450, 500, 550, 600, 650, or 700 μL is injected. In an implementation, the intermediate purity saponin extract is dissolved in a mixture of organic solvent and water at 40 mg/mL and 600 μL (24 mg) is injected into a preparative HILIC column.

Many different types of solid phases may be used with HILIC chromatography. For example, HILIC chromatography may be performed with an unbonded or surface-functionalized silica solid phase or a polymer solid phase. The solid phase may be surface-functionalized with diol, amide, amino, zwitterionic, or hybrid amide-amino groups. In an implementation, the solid phase may comprise core-shell silica microparticles. In an implementation, the solid phase may comprise amino-propyl silane bonded to silica microparticles. In an implementation, the solid phase may comprise silica microparticles coated with sulfobetaine groups or phosphorylcholine groups. In an implementation, the solid phase may comprise an amide polyol, amino group with linker and polar end-capping. In an implementation, the solid phase may have a particle size of 1.7, 2.6, 3, 4, 5, 6, 7, 8, 9, or 10 μm. In an implementation, particles in the solid phase may have a pore size of 60, 70, 80, 90, 100, 110, 120, 130, 140, 150, 160, 170, 180, 190, 200, or 210 Å.

For example, HILIC chromatography may be performed on a Kinetex® HILIC column, a TSKgel®-Amide 80 column, a Zorbax® NH₂ column, a Luna® NH₂ column, a Luna® Omega SUGAR column, or a Luna® HILIC column.

For example, HILIC chromatography may be performed on a Syncronis HILIC column or on a Sequant® ZIC®-HILIC column. Without being bound by theory, it is believed that the pH-independent presence of both positive and negative charges on a zwitterionic HILIC column may improve separation of mixtures of saponins.

In an implementation, the mobile phase comprises water and an organic solvent. The organic solvent may be acetonitrile, acetone, methanol or isopropanol. In an implementation, the mobile phase comprises a volatile salt. The volatile salt may be ammonium formate, ammonium acetate, formic acid, acetic acid, ammonium hydroxide, or ammonium carbonate. Formic acid or acetatic acid are more commonly used for low pH mobile phases. Ammonium hydroxide or ammonium carbonate are more commonly used for high pH mobile phases.

The HILIC chromatography may be performed under isocratic conditions or under a solvent gradient (e.g., continuous or stepped). In an implementation under isocratic conditions, the mobile phase may comprise 20-50% water. In an implementation, the isocratic conditions may include 10, 15, 20, 25, 35, 45, or 50% water. In an implementation, the solvent gradient may include 10-50% water such as, for example, a shallow gradient centered around 20% water.

Previous work indicates that hydrolysis of the ester bond linking the fatty acyl chain to the fucose, causing de-acylation of QS-21, is minimized around pH 5.5. (J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28.) Accordingly, in some implementations, HILIC chromatography may be performed at about pH 5.5. However, effective HILIC chromatography separation of saponins is not limited to a narrow pH range. HILIC chromatography may be performed at any pH between about 3.0-8.0, between about 3.0-6.0, between about 5.0-6.0. For example, HILIC chromatography may be performed at a pH of 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In an implementation, the runtime of the HILIC chromatography step until elution the peak containing QS-21 may be less than 7, 8, 9, 10, 11, 12, 13, 14, 15, 16, 17, 18, 19, 20, 21, 22, 23, 24, 25, 26, 27, 28, 29, 30, 31, 32, or 33 minutes on an analytical HILIC column at a flow rate of 1 mL/min. In an implementation, the runtime of the HILIC chromatography step until elution the peak containing QS-21 may be less than 18, 19, or 20 minutes on a preparative HILIC column at a flow rate of 20 mL/min.

In an implementation, fractions of eluate corresponding to one or more peaks in the chromatogram generated by HILIC chromatography may be collected and analyzed for saponin content. In an implementation, the saponin content may be analyzed by LC-MS. Fractions corresponding to a peak containing the saponin of interest may be collected. In an implementation, only fractions corresponding to the peak itself are collected. Fractions corresponding to left and right shoulder peaks may be excluded. The collected fractions following separation by HILIC chromatography contain a substantially pure saponin.

In an implementation, the substantially pure saponin may be dried and lyophilized. Drying may be performed by evaporating the organic solvent under a stream of nitrogen or filtered air. The dried product may then be lyophilized. to remove the water content. The dried product may be stored frozen at, for example, −20 or −80° C. The substantially pure saponin may be used as an adjuvant.

### F. Characterization of the Substantially Pure Saponin

Purity of a compound separated by chromatography may be determined by calculating the percent area under the curve (AUC) of the peak or peaks containing the saponin of interest. The y-axis of chromatograms show a measure of the intensity of absorbance (in units of mAU, or milli-Absorbance Units) or a normalized amount of absorbance. The x-axis is in units of time (typically minutes) and is used to determine the retention time for each peak. Techniques for calculating percent AUC are known to those of ordinary skill in the art and can be performed directly from the chromatogram by comparing the peak areas for the peak of interest compared to all other peaks in the chromatogram. This is usually expressed as the percentage of the total peak area represented by the peak area of interest. Total peak areas are calculated by area under the curve of the UV trace to its baseline. The baseline is the portion of the chromatogram recording the detector response when only the mobile phase emerges from the column.

The HPLC analytical methods for measuring purity on a C4 column described in this disclosure are designed to use UV absorption at a given wavelength for recording the presence and the amount of a compound in a sample passing the detector at any given point in time. For example, the primary output of any HPLC run with standard equipment will be an area percentage of the respective peak in the UV detection chromatogram, i.e., AUC. Particularly in the absence of any detailed information on specific extinction coefficients of the compound(s) present in a sample, the percent area values obtained by HPLC are typically equated with a “% by weight” value without applying any correction factor. For example, the AUC percent value for a single peak (eluted at a certain retention time) is assumed to correspond to the percent proportion of the compound by weight.

Characterization of a saponin may also be performed by mass spectrometry (MS). MS is an analytical technique used to identify and/or quantify molecules in a sample by sorting and detecting ionized molecules according to their mass-to-charge ratio (m/z). The results are typically presented as a mass spectrum, a plot of ion intensity as a function of m/z. Liquid chromatography-mass spectrometry (LC/MS or LC-MS) separates compounds chromatographically before they are introduced to the mass spectrometer. The LC mobile phase is usually a mixture of water and organic solvents with a small amount of a suitable additive. The additive can be ammonium formate, ammonium acetate, formic acid, acetic acid, or trifluoroacetic acid. Available LC-MS ionization methods include electrospray ionization, atmospheric pressure chemical ionization (APCI), and atmospheric pressure photoionization.

The term ‘m/z’ refers to the mass to charge ratio of an ion peak in a mass spectrum. Unless otherwise specified, ‘m/z’ is determined by liquid chromatography-mass spectrometry (LC/MS). The mass spectra show signal intensity versus m/z. In implementations, LC/MS may be performed on an LC/triple quadrupole (QqQ) system, an LC/quadrupole time-of-flight (Q-TOF) system, an LC/single quadrupole system, an LC/TOF system, an LC/ion trap system, or an LC/orbitrap system. Ion abundance is measured as ion counts shown on the y-axis of the mass spectrum. The most abundant ion or most abundant species refers to the single species that has an ion count greater than any other species shown in the same mass spectrum.

There are multiple forms of QS-21 that can be detected as distinct species by MS run in negative ion mode in a sample of Quillaja saponaria saponin extract. Mass-to-charge ratios (m/z) and descriptions of some forms of QS-21 are presented in the following table. M/z presented in this disclosure as integer values include ±m/z 0.5. For example, m/z 1000 includes the range of m/z values from 999.5 to 1000.5 inclusive of the endpoints.

| Description | m/z |
|---|---|
| singly charged negative ion of QS-21 | 1989 |
| singly charged de-acylated QS-21 | 1512 |
| ammonium adduct of doubly charged QS-21 | 1012 |
| double charged QS-21 | 994 |
| doubly charged de-acylated QS-21 | 755 |

The singly charged, intact form of QS-21 includes only the singly charged negative ion of QS-21 with m/z 1989. Impurity mass spectral peaks include all peaks other than those representing a form of the saponin. For example, in the mass spectra provided in this disclosure, impurity mass spectral peaks include all ion peaks other than those shown in the table above.

### G. Adjuvant Activity

The term “adjuvant activity” means the ability of a compound when administered to an individual or tested in vitro to increase the immune response to an antigen. Antibody titer is one way of measuring adjuvant activity. Increase in cell-mediated immune response is another way of measuring adjuvant activity. The adjuvant activity of saponins may be determined by any of a number of methods known to those of ordinary skill in the art as described in U.S. Pat. No. 5,057,540. Illustrative tests for adjuvant activity of saponins are described in U.S. Pat. No. 7,049,302.

Saponins are generally known to possess adjuvant activity. Quillaja saponins, particularly QS-7, QS-17, QS-18, and QS-21, have been found to be excellent stimulators of antibody response to soluble T-dependent protein antigens, “subunit antigens,” which are poorly immunogenic and require a potent adjuvant for maximization of immune responses. Examples of purified subunit antigens for which saponin adjuvants will augment the IgG response in mice include keyhole limpet hemocyanin (KLH), HIV-1 gp120 (Bomford, R. et al, AIDS Res. Hum. Retroviruses 821765 (1992)), and influenza nucleoprotein (Brett, S. et al., Immunology 802306 (1993)). QS-7, QS-17, QS-18 and QS-21 have also been shown to stimulate potent antibody responses in mice to the antigens bovine serum albumin and cytochrome b5 (Kensil, C. R. et al., J. Immunol. 1462431 (1991)). The level of antibody response induced by these purified saponins is comparable to other commonly used adjuvants, e.g., complete Freund's adjuvant, and superior to aluminum hydroxide.

QS-21 has also been shown to enhance antibody responses to T-independent antigens, including unconjugated bacterial polysaccharides (White, A. C. et al., “A purified saponin acts as an adjuvant for a T-independent antigen, in: Immunobiology of Proteins and Peptides, Vol. VI (M. Z. Atassi, ed.), Plenum Press, New York, pp. 207-210 (1991)). The immunogenicity of the vaccine was further increased by conjugating diphtheria toxoid to the polysaccharide. QS-21 enhanced the antibody response to the polysaccharide as well as the carrier, including IgG2a, IgG2b, and IgG3 responses. (Coughlin, R. T. et al., Vaccine 13(1):17-21 (1995)).

The saponin extracts are known to exhibit adjuvant effects when administered over a wide range of dosages and a wide range of ratios to the antigen being administered. In one implementation, a saponin extract is administered in a ratio of adjuvant to immunogen (w/w) of 3.0 or less or 1.0 or less.

### H. Pharmaceutical Compositions and Vaccine Compositions

Saponin extracts may be incorporated into a pharmaceutical composition or a vaccine composition. The saponin extract may be the substantially pure saponin extract of this disclosure. In illustrative implementations, the saponin may be QS-7, QS-17, QS-18, and/or QS-21. In an implementation, the saponin is substantially pure QS-21.

Pharmaceutical compositions generally comprise the substantially pure saponin extract in combination with a physiologically acceptable carrier. Pharmaceutical compositions may also include an antigen. The choice of suitable physiologically acceptable carrier will vary dependent upon the chosen mode of administration. “Pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art.

Vaccine compositions, also referred to as immunogenic compositions, comprise an antigen and a saponin such as the substantially pure saponin extract described in this disclosure. Generally, vaccine compositions are capable of producing an immune response to the antigen in a cell, tissue, or individual. In some implementations, a vaccine composition produces protective immunity against disease in individuals.

### I. Carriers and Excipients

The pharmaceutical compositions and vaccine compositions of the disclosure may be formulated using any of a variety of well-known procedures. In certain embodiments, the pharmaceutical compositions and vaccine compositions are prepared as stable emulsions (e.g., oil-in-water emulsions), liposomes or as aqueous solutions.

In certain applications, the compositions disclosed herein may be delivered via oral administration to a subject. As such, these compositions may be formulated with an inert diluent or with an assailable edible carrier, or they may be enclosed in a hard or soft-shell gelatin capsule, or they may be compressed into tablets, or they may be incorporated directly with the food of the diet.

In certain circumstances, it will be desirable to deliver the compositions disclosed herein parenterally, subcutaneously, intravenously, intradermally, intramuscularly, intranasally, intratracheally, or even intraperitoneally as described, for example, in U.S. Pat. Nos. 5,543,158, 5,641,515 and 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical composition forms suitable for injectable use include sterile aqueous solutions or dispersions and sterile powders for the extemporaneous preparation of sterile injectable solutions or dispersions (U.S. Pat. No. 5,466,468). In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts include the acid addition salts (formed with the free amino groups of the protein), which are formed with inorganic acids such as, for example, hydrochloric or phosphoric acids, or such organic acids as acetic, oxalic, tartaric, mandelic, and the like. Salts formed with the free carboxy groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known to one of ordinary skill in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an unacceptable allergic or similar untoward reaction when administered to a human. The preparation of an aqueous composition that contains a protein as an active ingredient is well understood to one of ordinary skill in the art. Typically, such compositions are prepared as injectables, either as liquid solutions or suspensions, solid forms suitable for solution in, or suspension in, liquid prior to injection can also be prepared. The preparation can also be emulsified.

In certain embodiments, the compositions of the present disclosure may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. Nos. 5,756,353 and 5,804,212. Likewise, the delivery of drugs using intranasal microparticle resins (Takenaga et al., 1998) and lysophosphatidyl-glycerol compounds (U.S. Pat. No. 5,725,871) are also well-known in the pharmaceutical arts. Likewise, transmucosal drug delivery in the form of a polytetrafluoroetheylene support matrix is described in U.S. Pat. No. 5,780,045.

A pharmaceutical composition or vaccine composition may, alternatively, contain an immunostimulant and a DNA molecule such that a desired polypeptide is generated in situ. In such compositions, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a particular embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells.

### J. Antigen

An antigen may be any target epitope, molecule (including a biomolecule), molecular complex (including molecular complexes that contain biomolecules), subcellular assembly, cell, or tissue against which elicitation or enhancement of immunoreactivity in a subject is desired. Frequently, the term antigen will refer to a polypeptide antigen of interest. However, antigen, as used herein, may also refer to a nucleic acid molecule (e.g., DNA or RNA) that encodes a polypeptide antigen. The antigen may also be a recombinant construct that encodes a polypeptide antigen of interest (e. g., an expression construct). Suitable antigens include, but are not limited to, a bacterial antigen, a viral antigen, a fungal antigen, a protozoan antigen, a plant antigen, a cancer antigen, or a combination thereto. The antigen described herein can be involved in, or derived from, for example, an infectious disease, cancer, autoimmune disease, allergy, asthma, or any other condition where stimulation of an antigen-specific immune response would be desirable or beneficial.

In certain implementations, the antigen may be derived from or is immunologically cross-reactive with at least one infectious pathogen that is associated with an infectious disease. In certain implementations, the antigen may be derived from or is immunologically cross-reactive with at least one epitope, biomolecule, cell, or tissue that is associated with cancer. In certain implementations, the antigen may be derived from or is immunologically cross-reactive with at least one epitope, biomolecule, cell, or tissue that is associated with an autoimmune disease.

Further, typical antigens suitable for use with the saponins of this disclosure include antigens derived from any of the following: viruses, such as influenza, SARS-CoV-2, feline leukemia virus, feline immunodeficiency virus, HIV-1, HIV-2, rabies, measles, hepatitis B, or hoof and mouth disease; bacteria, such as anthrax, diphtheria, Lyme disease, pneumococcus, or tuberculosis; or protozoans, such as Babeosis bovis or Plasmodium. The antigen may be a protein, a peptide, a polysaccharide, a lipid, a glycolipid, a phospholipid, or a nucleic acid encoding the antigenic protein or peptide of interest. The antigens may be purified from a natural source, synthesized employing solid phase synthesis, or may be obtained utilizing genetic engineering.

### K. Manufacture of a Medicament

A saponin extract may be used in the manufacture of a medicament, in particular as an adjuvant. The adjuvant may be administered separately from an immunogen or antigen, or may be combined, either during manufacturing or extemporaneously, with an immunogen or antigen as a medicament for combined administration. A medicament is a substance used for medical treatment. Examples of medicaments include pharmaceutical compositions and vaccine compositions.

Saponin extracts may be combined with further adjuvants, such as a TLR4 agonist, in particular lipopolysaccharide TLR4 agonists, such as lipid A derivatives, especially a monophosphoryl lipid A e.g. 3-de-O-acylated monophosphoryl lipid A (3D-MPL). monophosphoryl lipid A (3D-MPL). 3D-MPL can be produced according to the methods described in GB 2 220211A. Chemically it is a mixture of 3-deacylated monophosphoryl lipid A with 4, 5, or 6 acylated chains.

Other TLR4 agonists which may be of use with saponin extracts include Glucopyranosyl Lipid Adjuvant (GLA) such as described in WO 2008/153541 or WO 2009/143457 or in Coler R. N. et al. (2011) Development and Characterization of Synthetic Glucopyranosyl Lipid Adjuvant System as a Vaccine Adjuvant. PLoS ONE 6(1) and Arias M. A. et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41 144. WO 2008/153541 or WO2009/143457 identify TLR4 agonists which may be of use with saponin extracts.

A number of studies discuss the use of Quillaja saponins, particularly QS-21, in conjunction with other adjuvants. For example, QS-21 was shown to be an effective co-adjuvant with aluminum hydroxide (alum)-absorbed antigens. (Ma, J.-Y. et al., Vaccine 12(10):925-933 (1994); Newman, J. et al., J. Immunol. 148(8):2357-2362 (1992); Kensil, C. R. et al., “Structural and Immunological Characterization of the Vaccine Adjuvant QS-21,” in Vaccine Design: The Subunit and Adjuvant Approach, Powell, M. F. and Newman, M. J. eds., Plenum Press, New York (1995); Kensil et al., J. Am. Vet. Med. Assoc. 199:1423-1427 (1991); Wu, J.-Y. et al., J. Immunol. 148:1519-1525 (1992); Kensil et al., Vaccine Res. 2:273-281 (1993)).

A typical adult human dose of adjuvant may contain a saponin extract at amounts between 1 and 100 μg per dose. The saponin extract may be used at a level of about 50 μg. Examples of suitable ranges are 40-60 μg, 45-55 μg, or 49-51 μg. In a further implementation, the human dose comprises saponin extract at a level of about 25 μg. Examples of lower ranges include 20-30 μg, 22-28 μg, or 24-26 μg. Human doses intended for children may be reduced compared to those intended for an adult (e.g. reduction by 50%).

### L. Methods of Eliciting or Enhancing an Immune Response

Provided herein are methods of eliciting or enhancing an immune response in a subject, including the step of administering to a subject in need thereof a liposomal formulation, a pharmaceutical composition, or a vaccine composition described herein. In some embodiments, the formulations or compositions further comprise an antigen where the antigen is a polypeptide antigen or a nucleic acid molecule encoding a polypeptide antigen. In some such embodiments, the formulations or compositions are suitable for mixing with a polypeptide antigen or a nucleic acid molecule encoding a polypeptide antigen.

In the embodiments provided herein, the subject is a mammal (e.g., an animal including farm animals (cows, pigs, goats, horses, etc.), pets (cats, dogs, etc.), and rodents (rats, mice, etc.), or a human. In one embodiment, the subject is a human. In another embodiment, the subject is a non-human mammal. In another embodiment, the non-human mammal is a dog, cow, or horse.

In exemplary embodiments, the liposomal formulations disclosed herein are incorporated into vaccine compositions. The liposomal formulations described herein can be used for eliciting or enhancing an immune response in the subject (including a non-specific response and an antigen-specific response). In some embodiments, the immune response comprises a systemic immune response. In some embodiments, the immune response comprises a mucosal immune response. Elicitation or enhancement of an immune response includes stimulating an immune response and boosting an immune response.

The disclosure thus provides compositions for altering (i.e., increasing or decreasing in a statistically significant manner, for example, relative to an appropriate control as will be familiar to persons skilled in the art) immune responses in a host capable of mounting an immune response. As will be known to persons having ordinary skill in the art, an immune response may be any active alteration of the immune status of a host, which may include any alteration in the structure or function of one or more tissues, organs, cells, or molecules that participate in maintenance and/or regulation of host immune status. Typically, immune responses may be detected by any of a variety of well-known parameters, including but not limited to in vivo or in vitro determination of soluble immunoglobulins or antibodies, soluble mediators such as cytokines, lymphokines, chemokines, hormones, growth factors, and the like as well as other soluble small peptide, carbohydrate, nucleotide, and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death), or any other criterion by which the presence of an immune response may be detected. Accordingly, the formulations can act to enhance and/or induce antibody production, (e.g., induce production of neutralizing antibodies, enhance antigen specific antibody responses).

Immune responses may often be regarded, for instance, as discrimination between self and non-self structures by the cells and tissues of a host's immune system at the molecular and cellular levels, but the disclosure should not be so limited. For example, immune responses may also include immune system state changes that result from immune recognition of self molecules, cells or tissues, as may accompany any number of normal conditions such as typical regulation of immune system components, or as may be present in pathological conditions such as the inappropriate autoimmune responses observed in autoimmune and degenerative diseases. As another example, in addition to induction by up-regulation of particular immune system activities (such as antibody and/or cytokine production, or activation of cell mediated immunity) immune responses may also include suppression, attenuation, or any other down-regulation of detectable immunity, which may be the consequence of the antigen selected, the route of antigen administration, specific tolerance induction or other factors.

Determination of the induction of an immune response by the vaccine compositions of the present disclosure may be established by any of a number of well-known immunological assays with which those having ordinary skill in the art will be readily familiar. Such assays include, but need not be limited to, to in vivo or in vitro determination of soluble antibodies, soluble mediators such as cytokines, lymphokines, chemokines, hormones, growth factors, and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; or cellular differentiation by cells of the immune system, including altered surface antigen expression profiles or the onset of apoptosis (programmed cell death). Procedures for performing these and similar assays are widely known and may be found, for example, in Lekovits (Immunology Methods Manual: The Comprehensive Sourcebook of Techniques, 1998, see also Current Protocols in Immunology, see also, e.g., Weir, Handbook of Experimental Immunology, 1986 Blackwell Scientific, Boston, Mass, Mishell and Shigii (eds) Selected Methods in Cellular Immunology, 1979 Freeman Publishing, San Francisco, Calif, Green and Reed, 1998 Science 281:1309 and references cited therein).

Another way of assessing the immunogenicity of the pharmaceutical compositions or vaccine compositions disclosed herein where the nucleic acid molecule encodes a protein antigen is to express the recombinant protein antigen for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within protein antigens.

Any number of other immunological parameters may be monitored using routine assays that are well known in the art. These may include, for example, antibody dependent cell-mediated cytotoxicity (ADCC) assays, secondary in vitro antibody responses, flow immunocytofluorimetric analysis of various peripheral blood or lymphoid mononuclear cell subpopulations using well-established marker antigen systems, immunohistochemistry, or other relevant assays. These and other assays may be found, for example, in Rose et al. (Eds.), Manual of Clinical Laboratory Immunology, 5^{th} Ed, 1997 American Society of Microbiology, Washington, DC.

The efficacy of the compositions provided herein can also be determined in vivo by challenging appropriate animal models with the pathogen of interest infection.

Typical routes of administration of the pharmaceutical composition and vaccine composition include, without limitation, oral, topical, parenteral, sublingual, buccal, rectal, vaginal, intravenous, intradermal, transdermal, intranasal, intramucosal, or subcutaneous. In some exemplary embodiments, administration of the stable emulsion, liposomal formulation, pharmaceutical composition, and vaccine composition is intramuscular, ocular, parenteral, or pulmonary.

### EXAMPLES

VET-SAP® was used as the starting material for all purification techniques described in this disclosure. It is to be understood that Quil-A® or any other partially enriched Quillaja saponaria extract could be used instead of VET-SAP®. Powdered VET-SAP® was dissolved in water at 100 mg/mL. The dissolved solution of VET-SAP® was filtered prior to loading onto chromatography columns with 250 mL, 0.2 μm polyethersulfone membrane disposable filter units (Corning Inc., Corning, NJ, USA).

The mobile phases used in the various examples described in this disclosure were created from commercially available reagents according to techniques known to those of skill in the art. Acetonitrile (MeCN, HPLC grade), formic acid (99.0+% Optima™ LC/MS grade), ammonium acetate (Crystalline/HPLC grade), ammonium formate (Crystalline/ACS grade) and glacial acetic acid (HPLC grade) were purchased from Fisher Scientific (Hampton, NH, USA). The 20 L NOWPak® purchased from MilliporeSigma (Burlington, Mass., USA) was used for organic solvent mobile phases containing acetonitrile with 0.1% formic acid. Water (H₂O) used in the examples of this disclosure was ultrapure water obtained from a Ultrapure Lab Water System (MilliporeSigma, Burlington, Mass., USA).

All analytical chromatography columns were run on a Shimadzu Prominence HPLC system (Shimadzu, Kyoto, Japan). All preparative chromatography columns were run on a Gilson GX-281 preparative HPLC system (Gilson, Middleton, Wis., USA). Both HPLC systems were operated according to manufacturer instructions using conditions indicated in the examples. All chromatography runs were performed at room temperature (about 22° C.).

The mass spectra presented in this disclosure were generated by liquid chromatography/mass spectrometry (LC/MS). LC/MS analyses were performed on either an Agilent Technologies 6460 Triple Quadrupole (QqQ) LC/MS system or an Agilent 6520 Quadrupole Time-of-Flight (Q-TOF) LC/MS system as indicated as LC/QqQ-MS or LC/Q-TOF-MS, respectively. Each of the two systems was equipped with an Agilent 1290 Infinity LC system.

LC/QqQ-MS analyses on the Agilent 6460 Triple Quadrupole system were run as follows: 10 μL of 1 mg/mL samples dissolved in 50:50 acetonitrile:water with 0.05 wt % ammonium acetate were injected into the LC module without a column and run isocratically in 50:50 acetonitrile:water with 0.05 wt % ammonium acetate at 0.2 mL/min. UV absorption was detected at 210 nm. MS was run in negative ion mode with electrospray ionization (ESI), 100-2,500 m/z, scan time 500, fragmentor 80, accelerator voltage 7 V, step size 0.1 amu, delta EMV(−) 50-200, gas temperature 350° C., gas flow rate 9 L/min, nebulizer 45 pounds per square inch gauge (psig), and capillary voltage 4,500 V.

LC/Q-TOF-MS analyses on the Agilent 6520 Quadrupole Time-of-Flight system were run as follows: Samples in the form of lyophilized powders were resuspended at 1 mg/mL in 50:50 acetonitrile:water. Twenty μL of each sample was injected into the LC module without a column and run isocratically in 50:50 acetonitrile:water with 0.05 wt % ammonium acetate at 0.1 mL/min. Elucidation of the composition of major and minor peaks in the substantially pure QS-21 was carried out by first separating the sample on a traditional RP analytical C4 column. Mobile phase A: H₂O with 0.05 wt % ammonium acetate, mobile phase B: MeCN with 0.05 wt % ammonium acetate, flow rate: 0.1 mL/min. Gradient condition: 0.0 min-35% B, 20.0 min-40% B, 21.0 min-40% B, 26.0 min-90% B, 34.0 min-90% B, 35.0 min-35% B. UV absorption was detected at 210 nm. MS was run in dual ESI negative ion mode, 200-2,500 m/z, scan rate 3 spectra/sec, gas temperature 350° C., gas flow rate 12 L/min, nebulizer 50 psig, capillary voltage 2,500 V, and fragmentor 145.

All lyophilization of collected fractions was performed on a VirTis FreezeMobile 25 EL (SP Scientific, Warminster, PA, USA) after evaporation of acetonitrile under a steady stream of nitrogen or filtered air. Dried samples may be frozen and stored at −20 to −80° C. or resuspended and used without freezing.

The following HPLC columns are used in the examples provided in this disclosure.

| Type | Name | Characteristics | Source |
|---|---|---|---|
| traditional | analytical Gemini ® C18 | 250 × 4.6 mm, 5 μm, 110 Å | Phenomenex |
| RP | | | (Torrance, CA, USA) |
| traditional | preparative Gemini ® | 250 × 21.2 mm, 5 μm, 110 Å | Phenomenex |
| RP | C18 | | |
| traditional | Vydac ® C4 | 250 × 4.6 mm, 5 μm, 300 Å | Avantor (Radnor |
| RP | | | Township, PA, USA) |
| traditional | preparative Polaris ™ C8 | 250 × 21.2 mm, 5 um, 110 Å | Agilent Technologies |
| RP | | | (Santa Clara, CA, |
| | | | USA) |
| polar RP | Synergi ™ Hydro RP | 250 × 4.6 mm, 4 μm, 80 Å | Phenomenex |
| polar RP | analytical Luna ® | 250 × 4.6 mm, 5 μm, 100 Å | Phenomenex |
| | Omega Polar C18 | | |
| polar RP | preparative Luna ® | 250 × 50 mm, 5 μm, 100 Å | Phenomenex |
| | Omega Polar C18 | | |
| HILIC - | Kinetex ® HILIC | 250 × 4.6 mm, 5 μm, 100 Å | Phenomenex |
| unbonded | | | |
| silica | | | |
| HILIC - | TSKgel ®-Amide 80 | 250 × 4.6 mm, 5 μm, 100 Å | TOSOH Bioscience |
| amide | | | (Tokyo, Japan) |
| HILIC - | Zorbax ® NH₂ | 250 × 4.6 mm, 5 μm, 70 Å | Agilent Technologies |
| amino | | | |
| HILIC - | Luna ® NH₂ | 250 × 4.6 mm, 5 μm, 100 Å | Phenomenex |
| amino | | | |
| HILIC - | analytical Syncronis ™ | 250 × 4.6 mm, 5 μm, 100 Å | Thermo Fisher |
| zwitterionic | HILIC | | Scientific (Waltham, |
| | | | MA, USA) |
| HILIC - | preparative Syncronis ™ | 250 × 21.2 mm, 5 μm, 100 Å | Thermo Fisher |
| zwitterionic | HILIC | | Scientific |
| HILIC - | Sequant ® ZIC ®-HILIC | 250 × 4.6 mm, 5 μm, 200 Å | MilliporeSigma |
| zwitterionic | | | (Burlington, MA, |
| | | | USA) |
| HILIC - | Luna ® Omega SUGAR | 250 × 4.6 mm, 3 μm, 100 Å | Phenomenex |
| amide/amino | | | |

### Example 1—Analytical Comparison of Polar RP to Traditional RP Columns

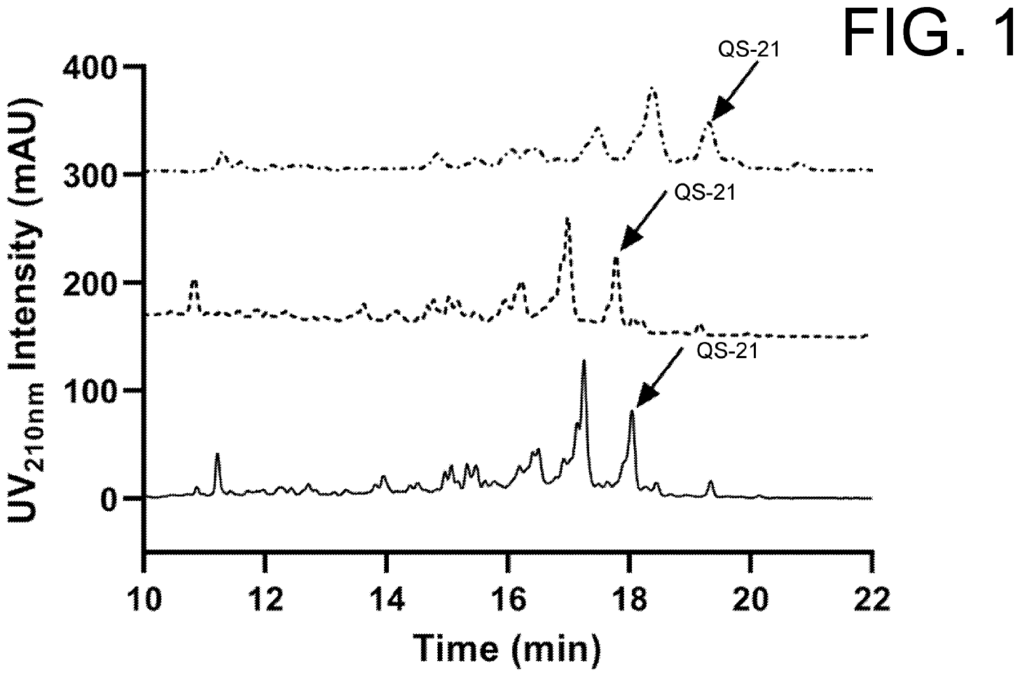

Two different polar RP columns were compared to each other and to a traditional RP column. The polar RP columns are a Synergi Hydro RP column (250×4.6 mm, 4 μm, 80 Å; shown with a dashed line) and a Luna Omega Polar C18 column. (250×4.6 mm, 5 μm, 100 Å; shown with a solid line). These are compared to a traditional RP column which is a Gemini C18 column (250×4.6 mm, 5 μm, 110 Å; shown with a dot dash line) in FIG. 1. VET-SAP® was dissolved in H₂O at 100 mg/mL without any pre-treatment and was separated by a 20-80% B gradient (mobile phase A: H₂O with 0.1% formic acid, mobile phase B: MeCN with 0.1% formic acid) on all three columns.

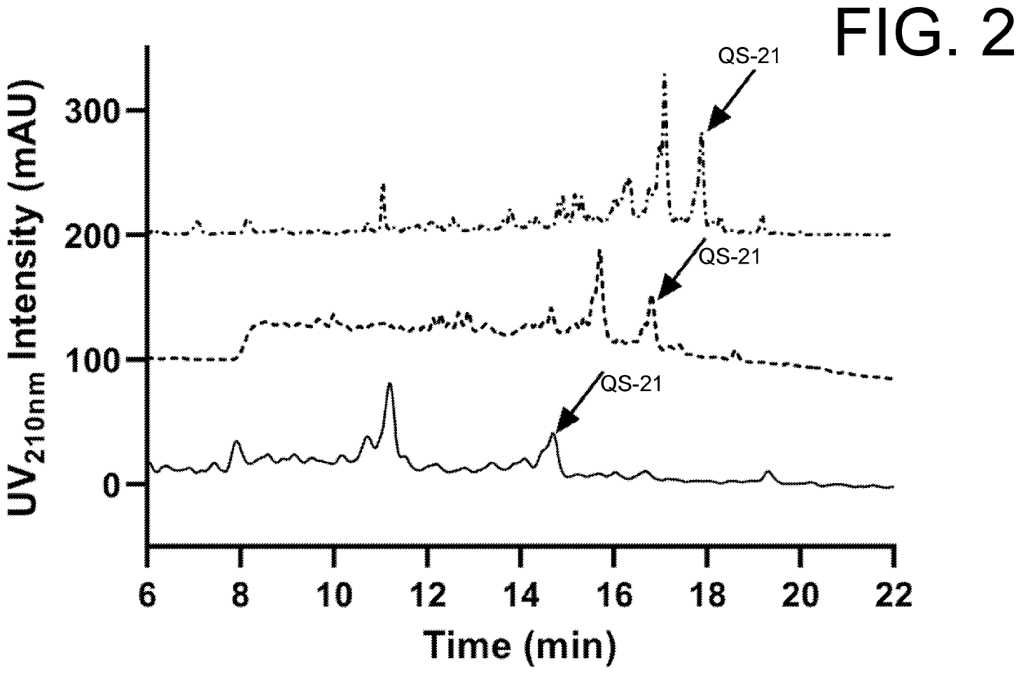

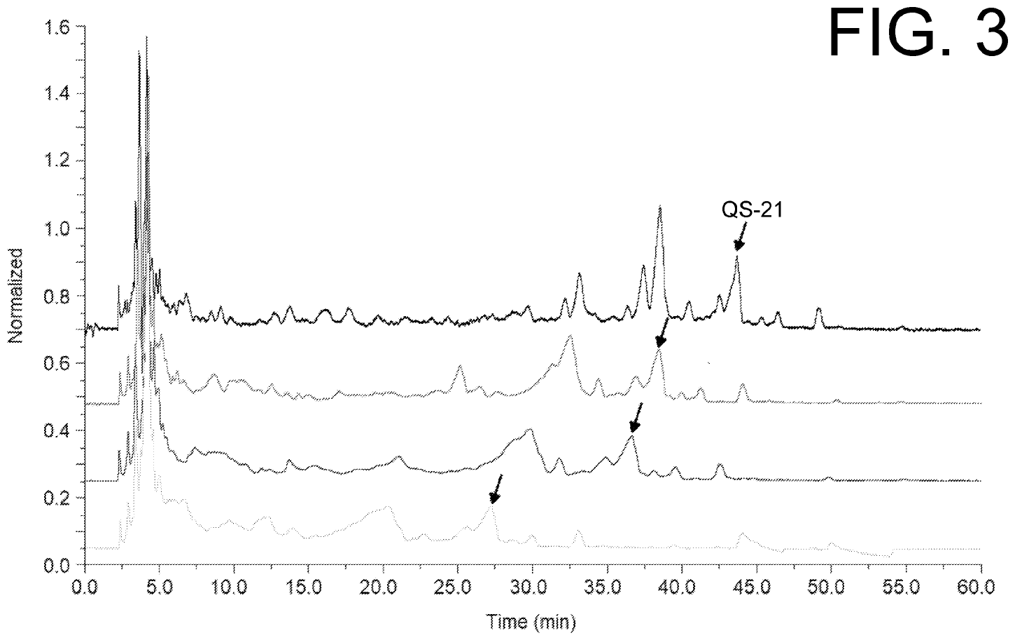

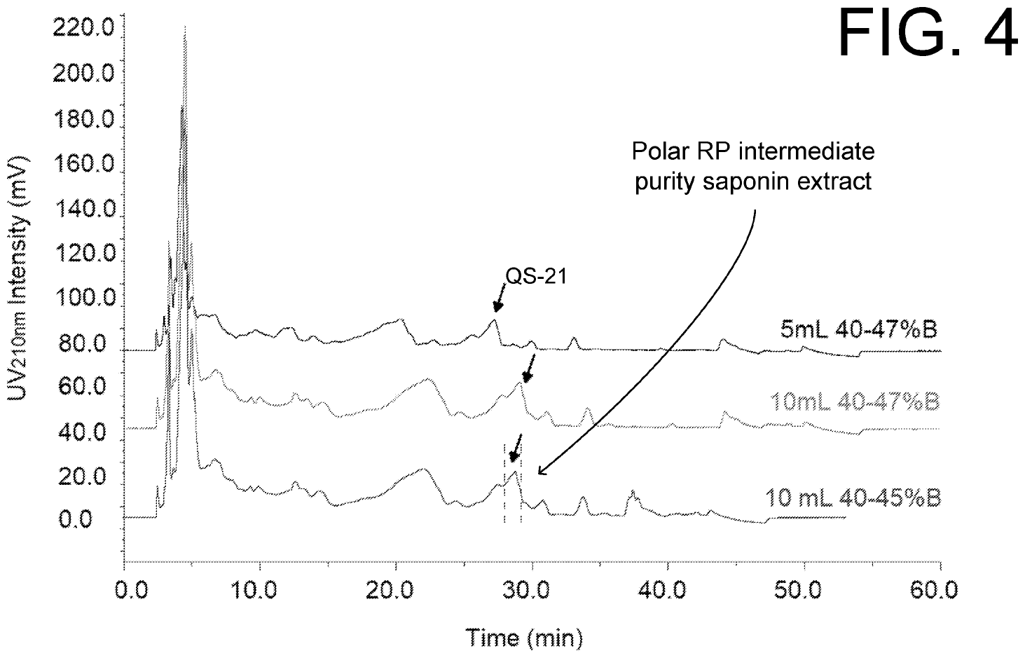

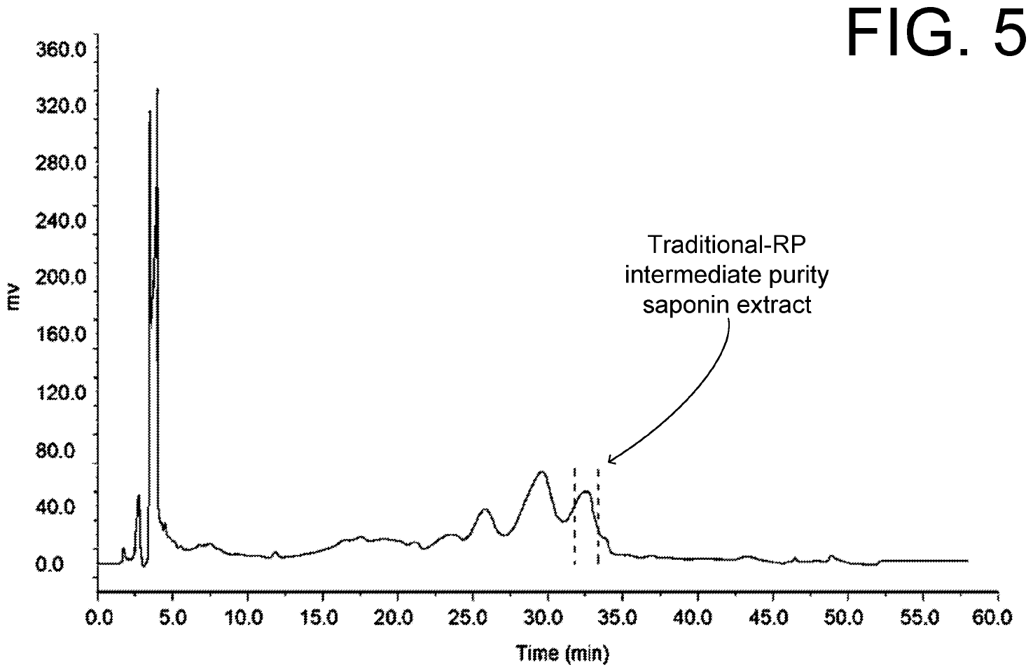

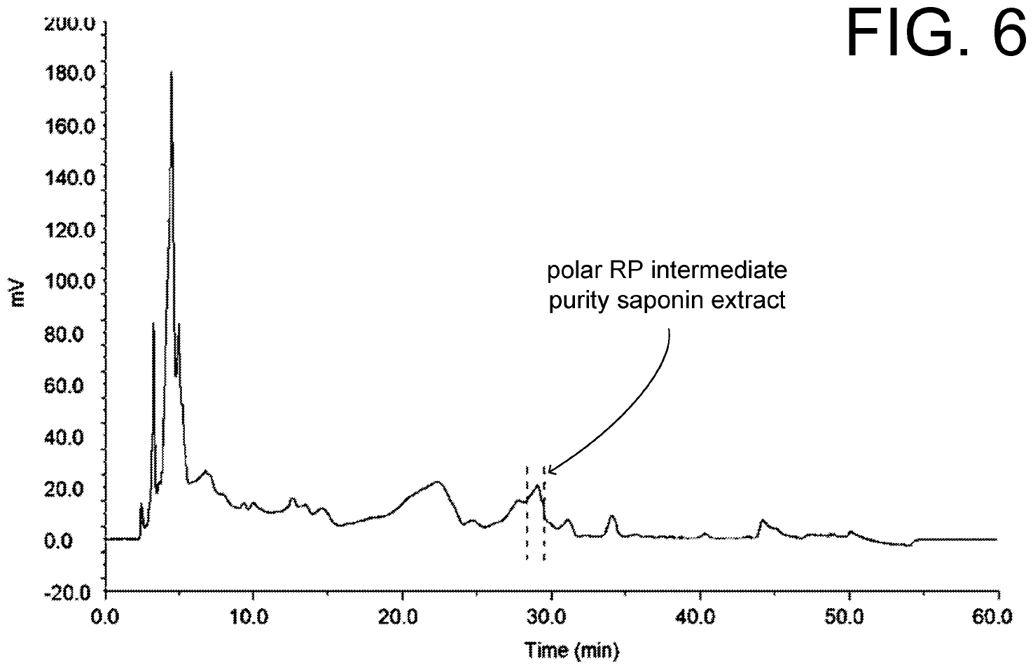

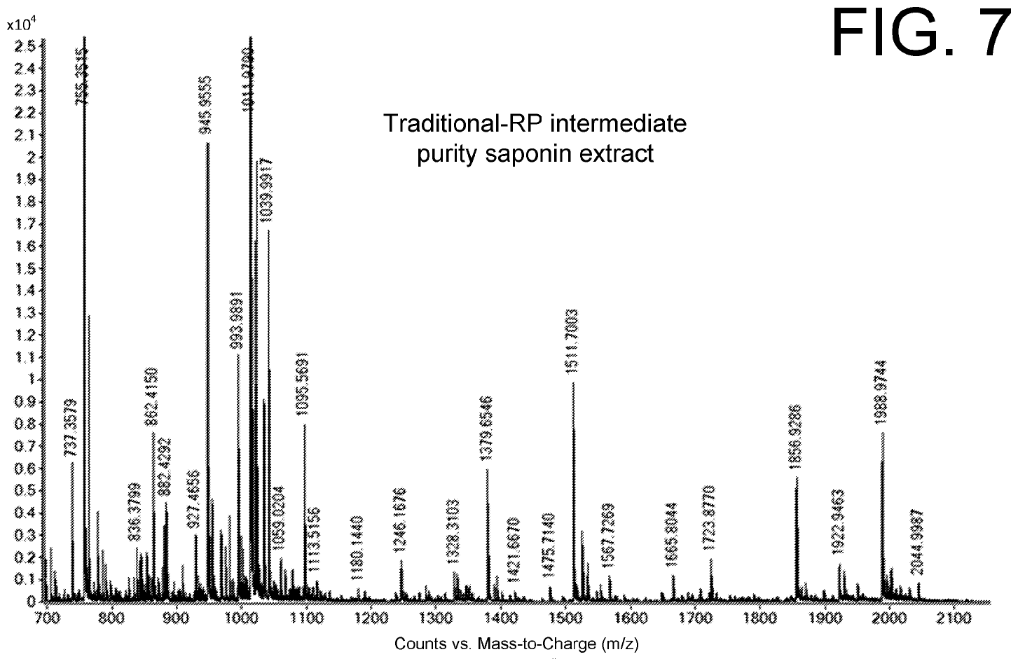

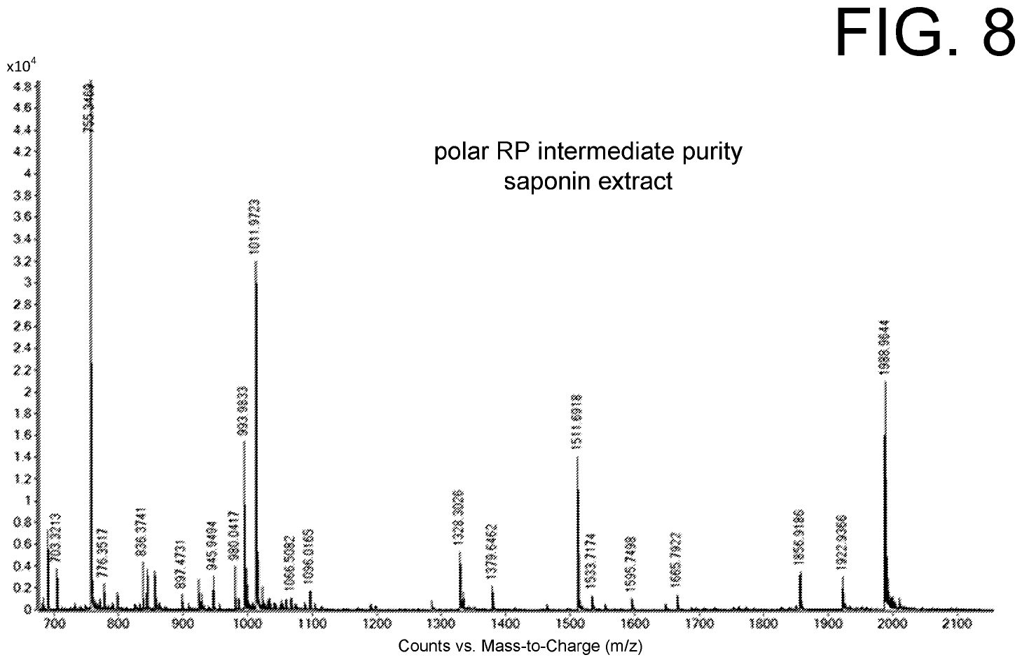

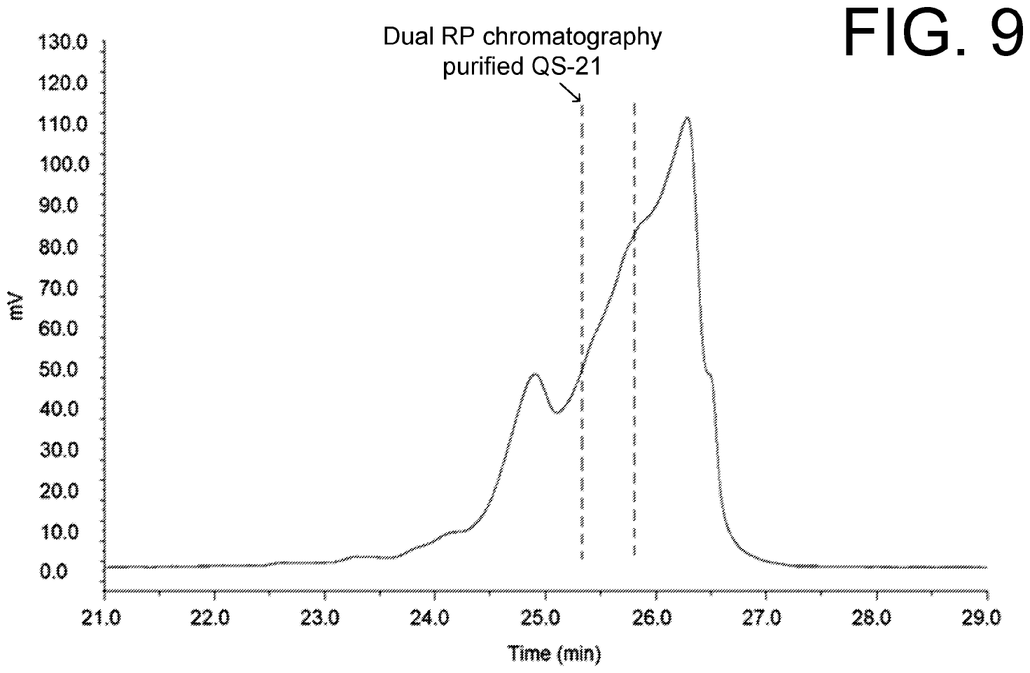

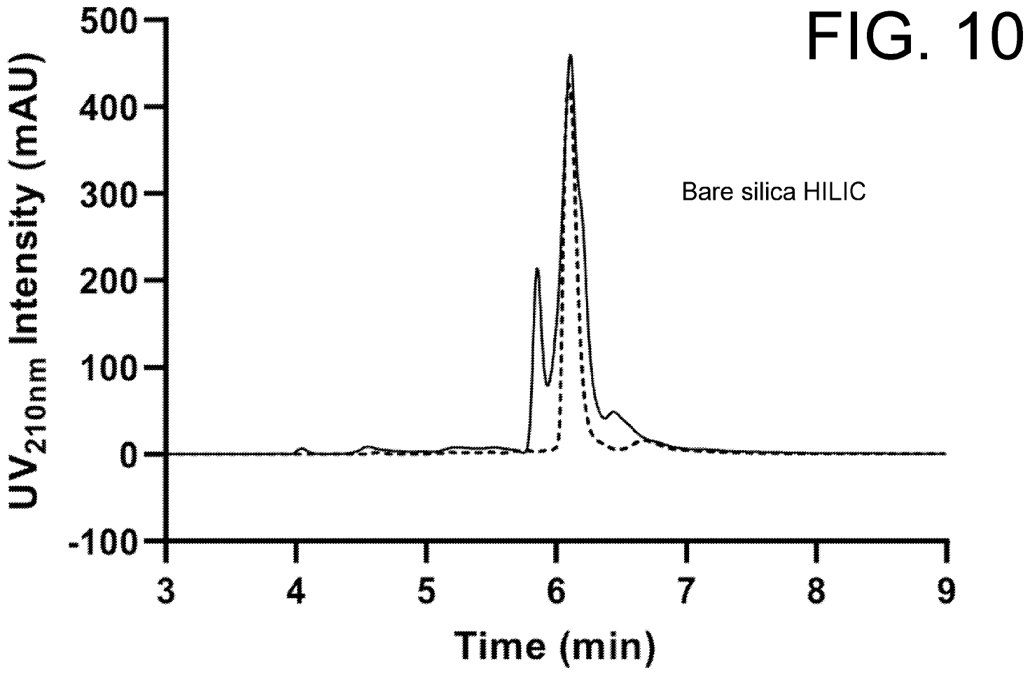

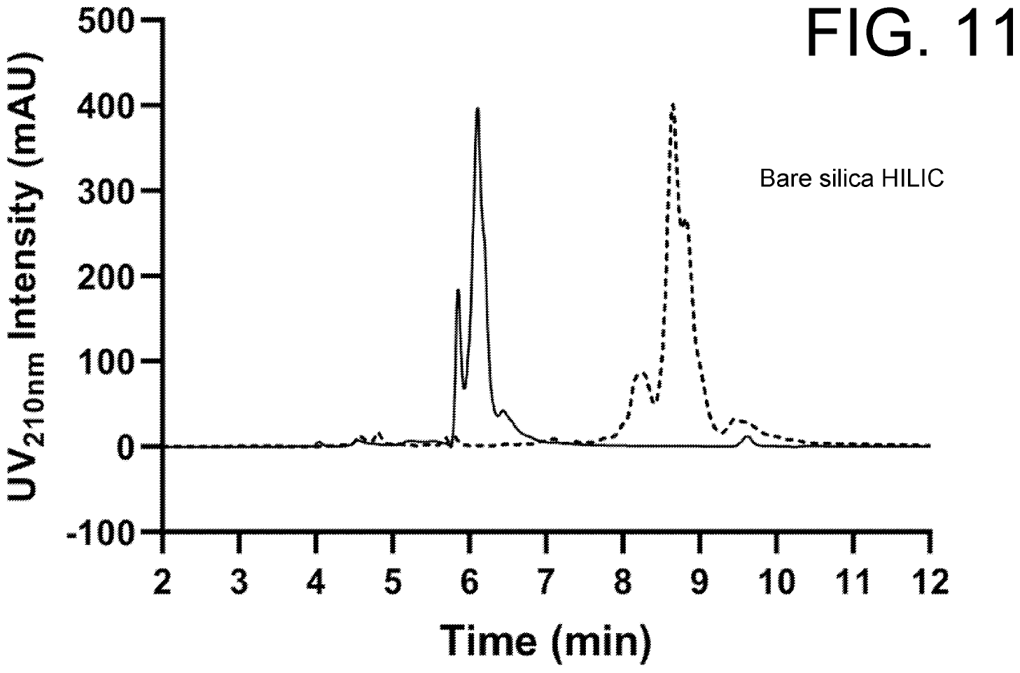

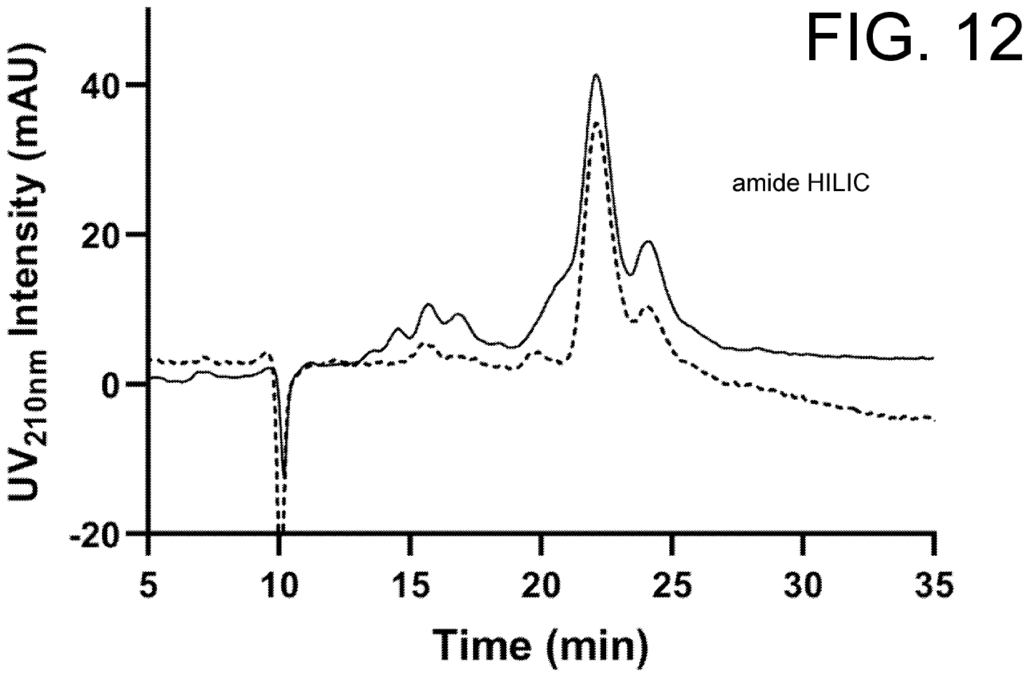

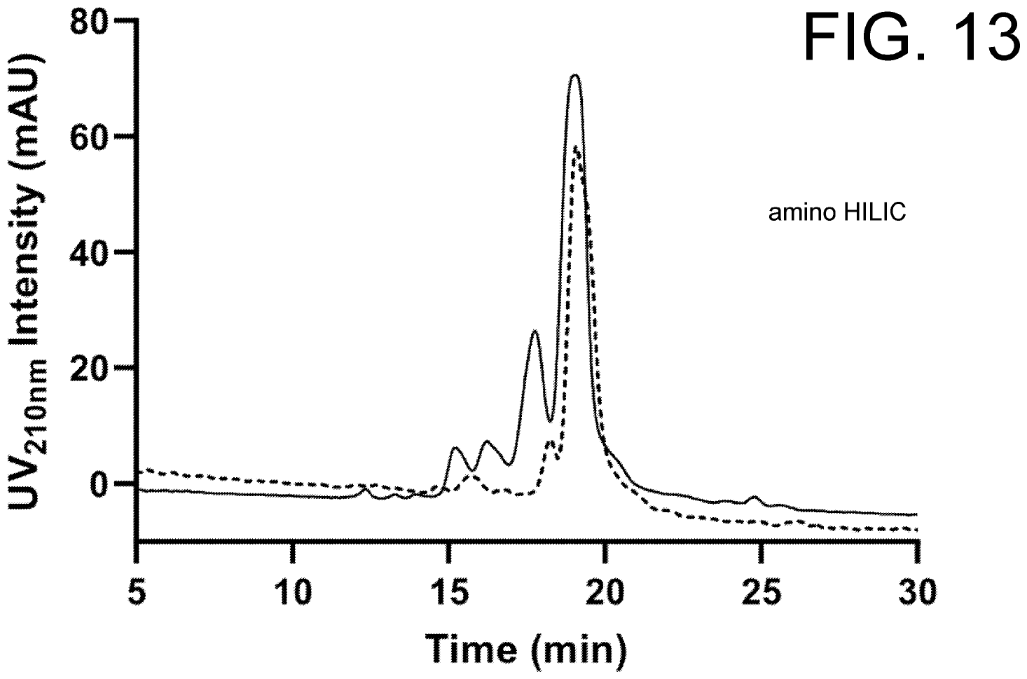

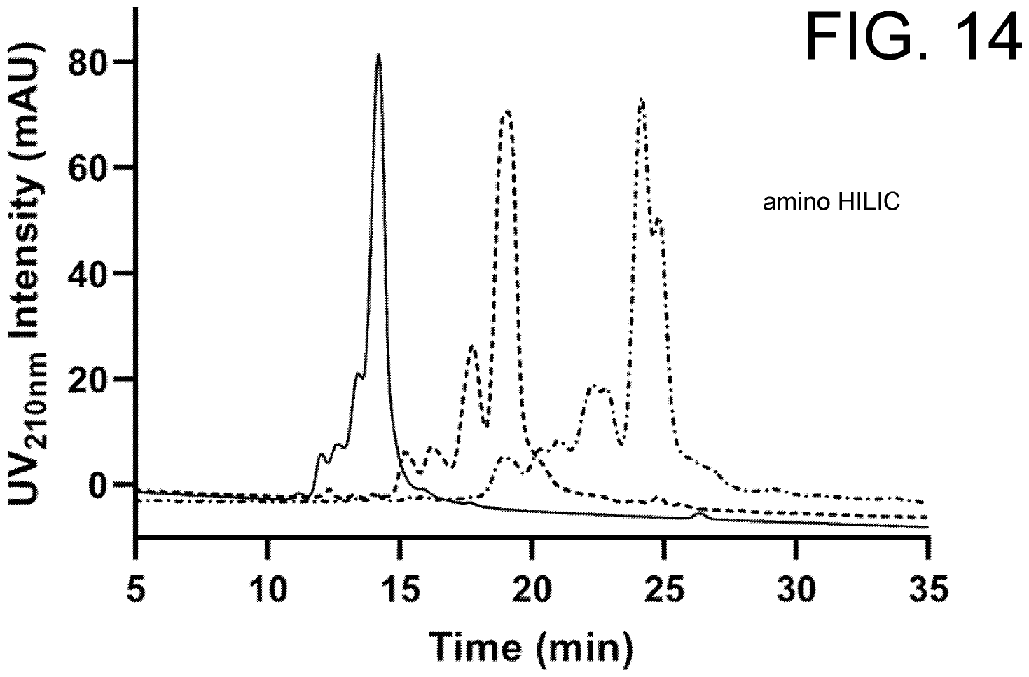

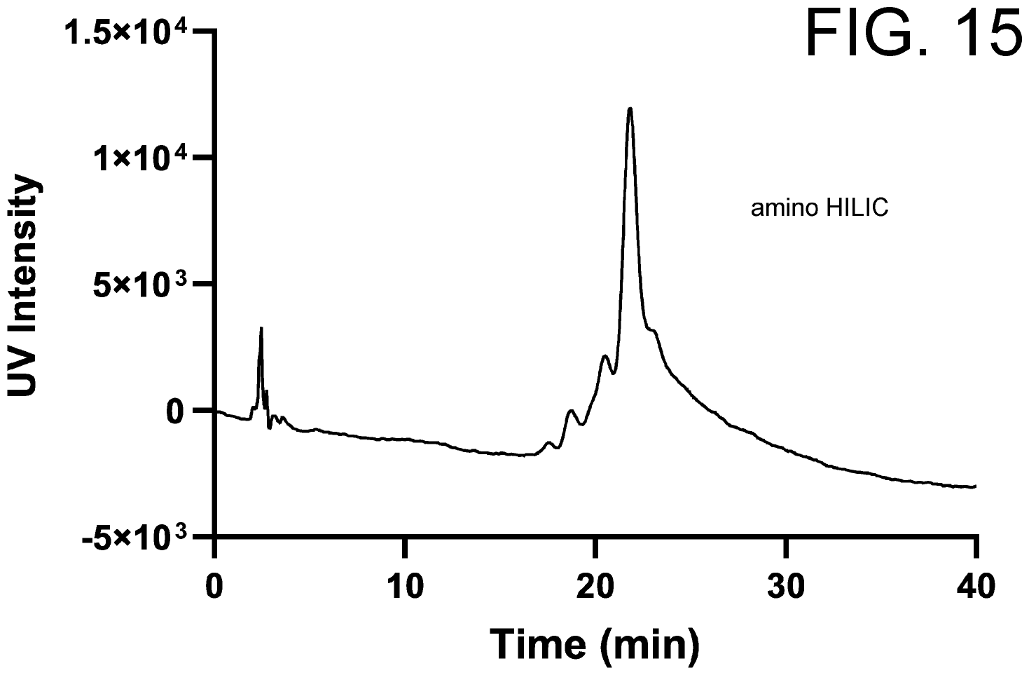

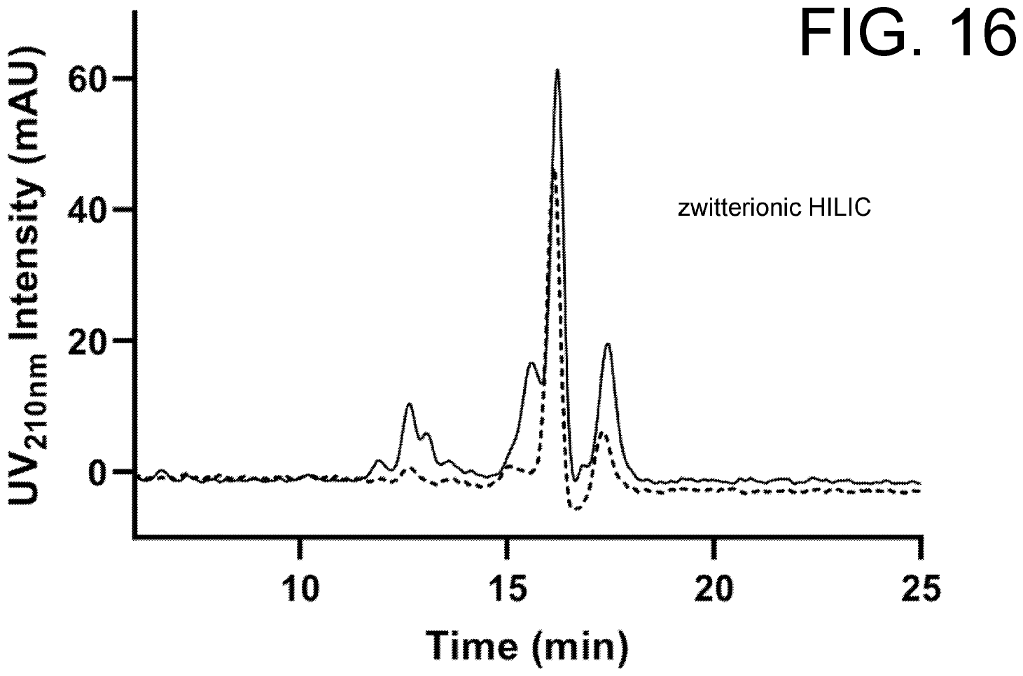

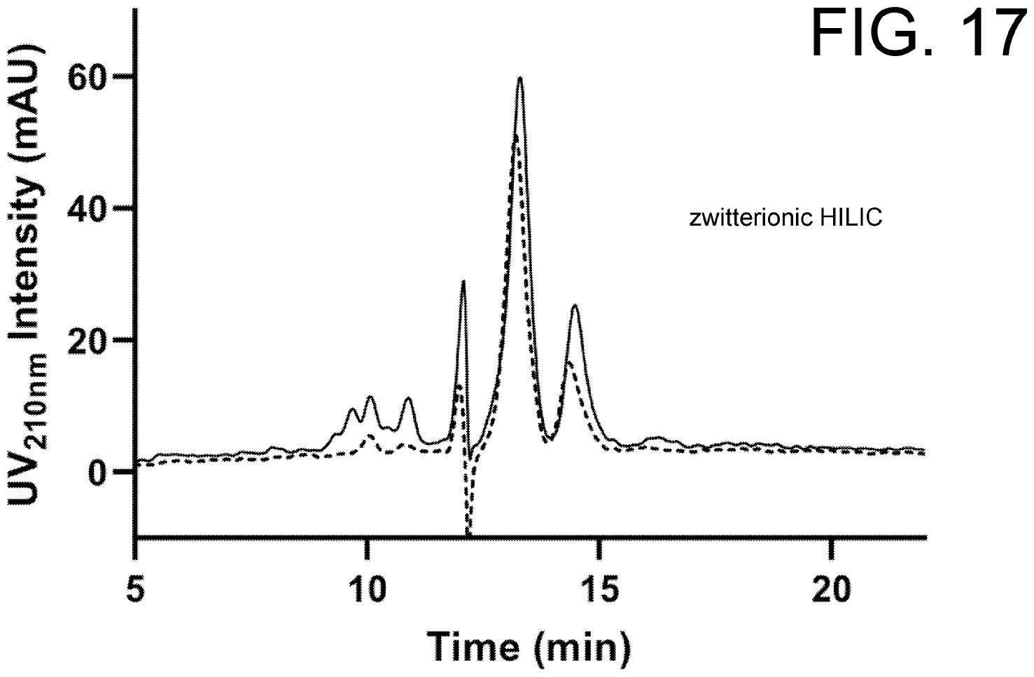

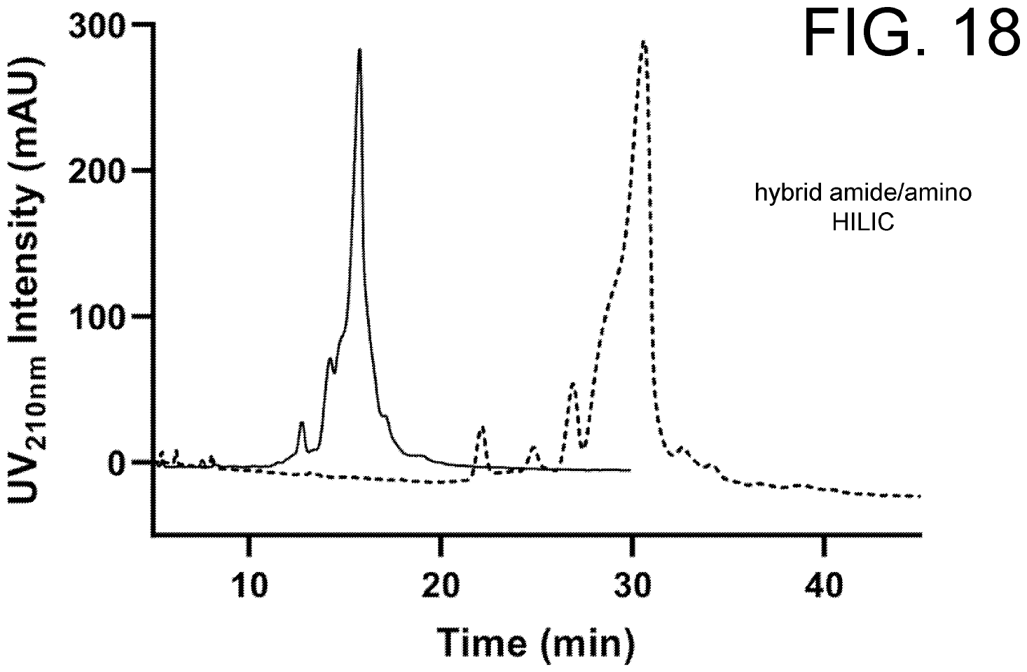

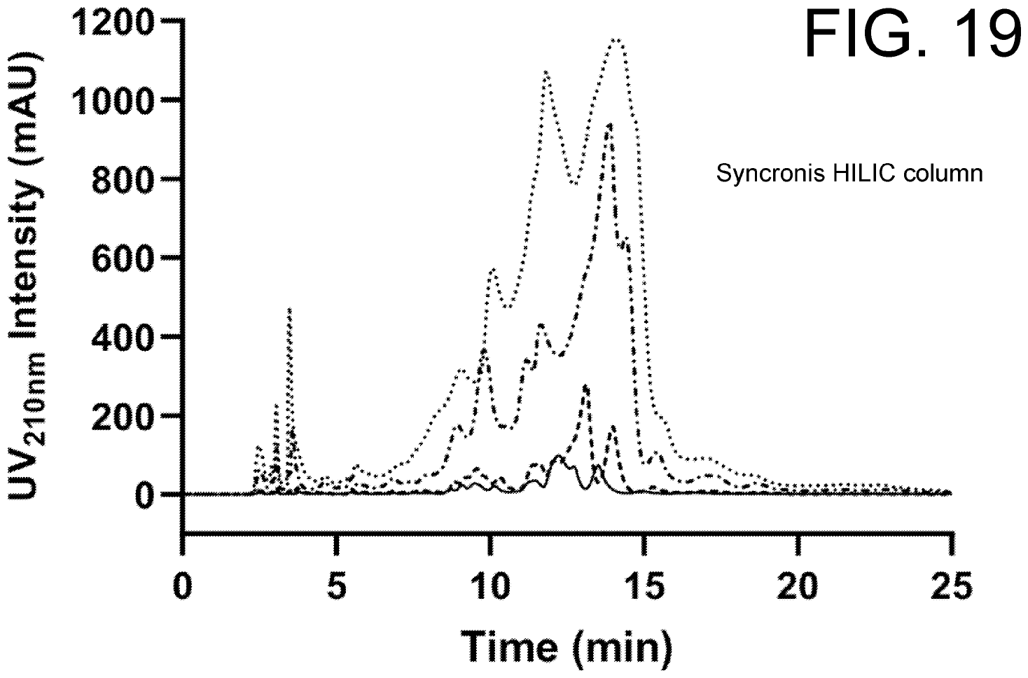

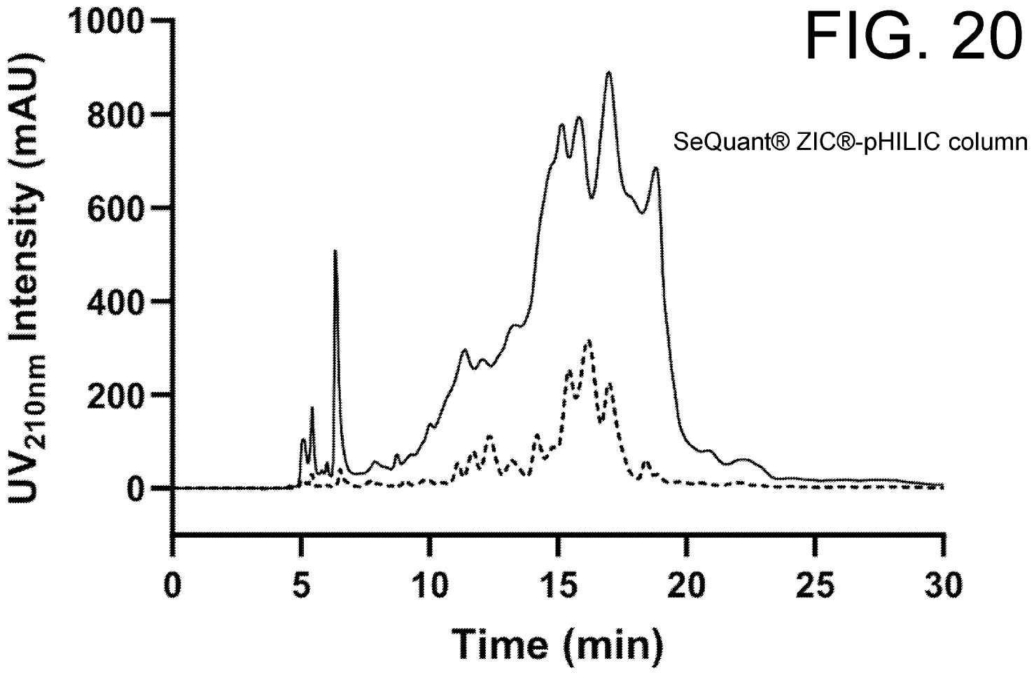

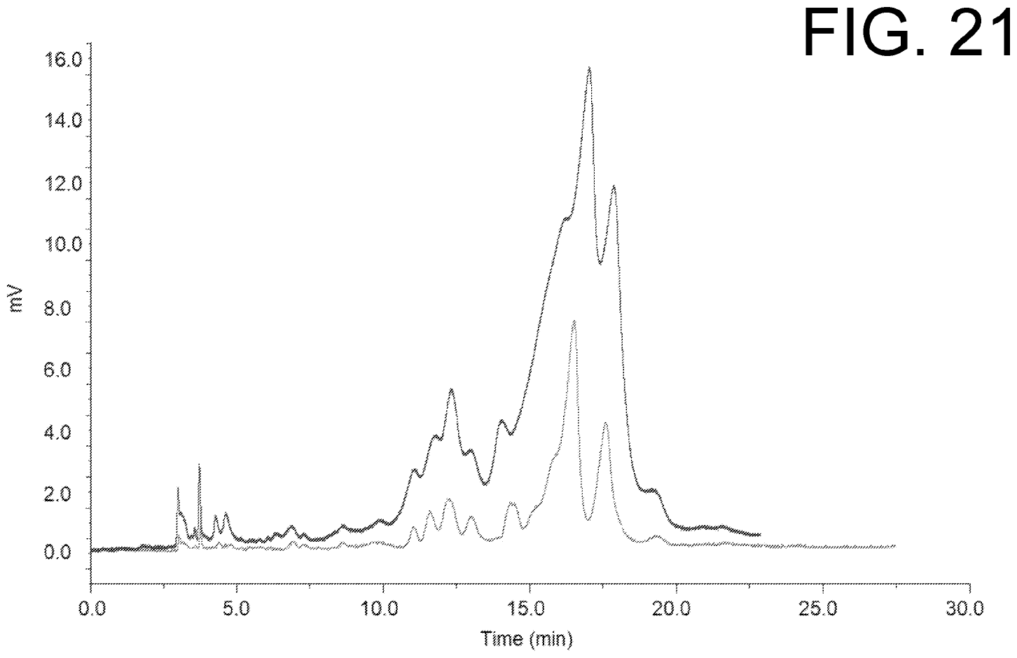

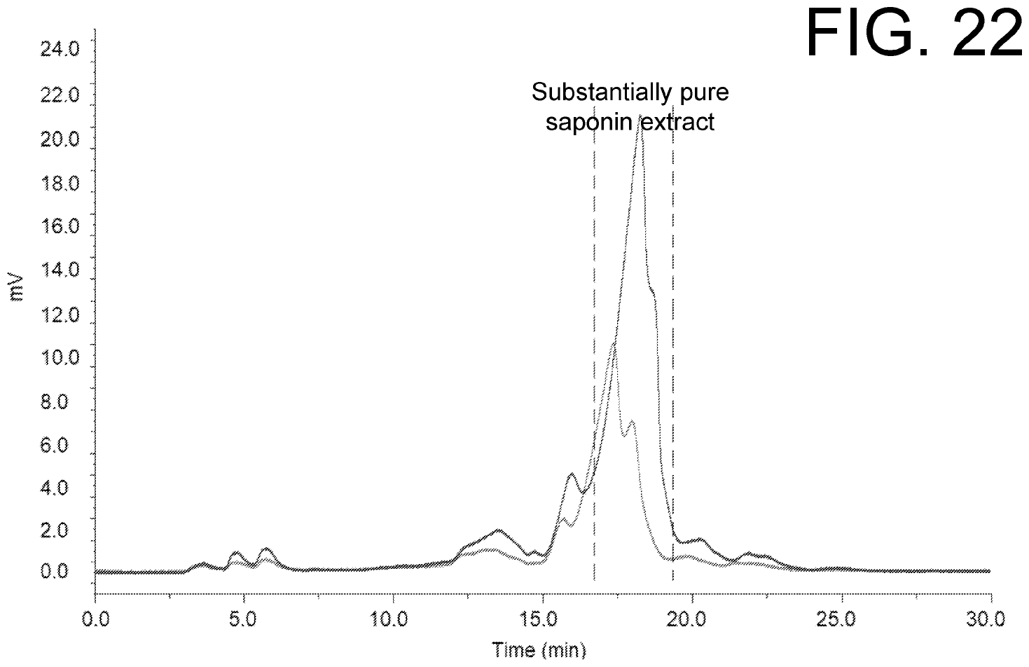

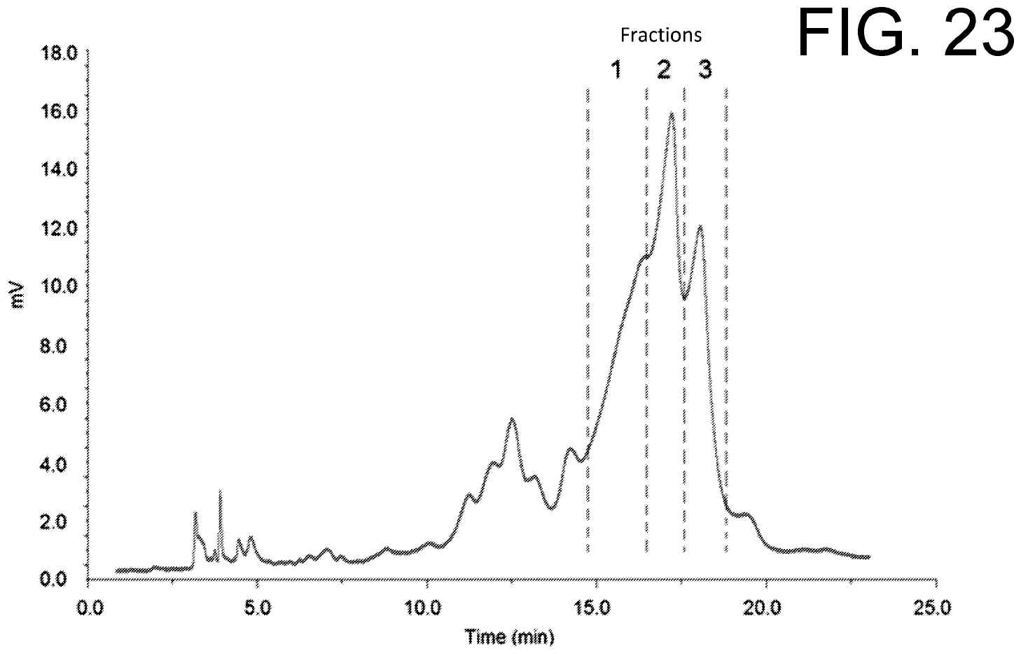

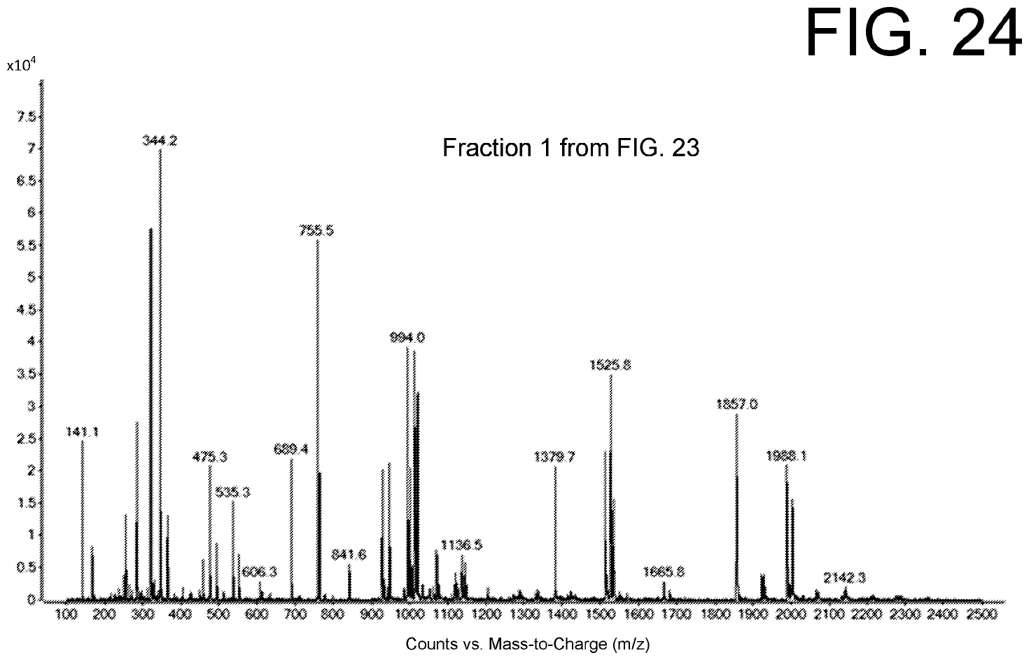

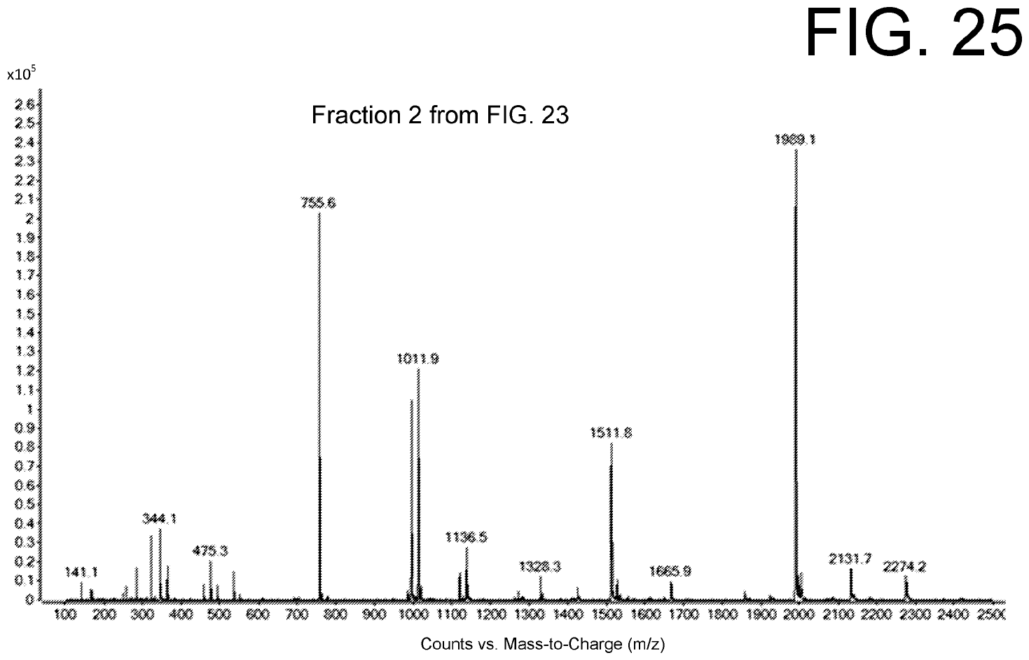

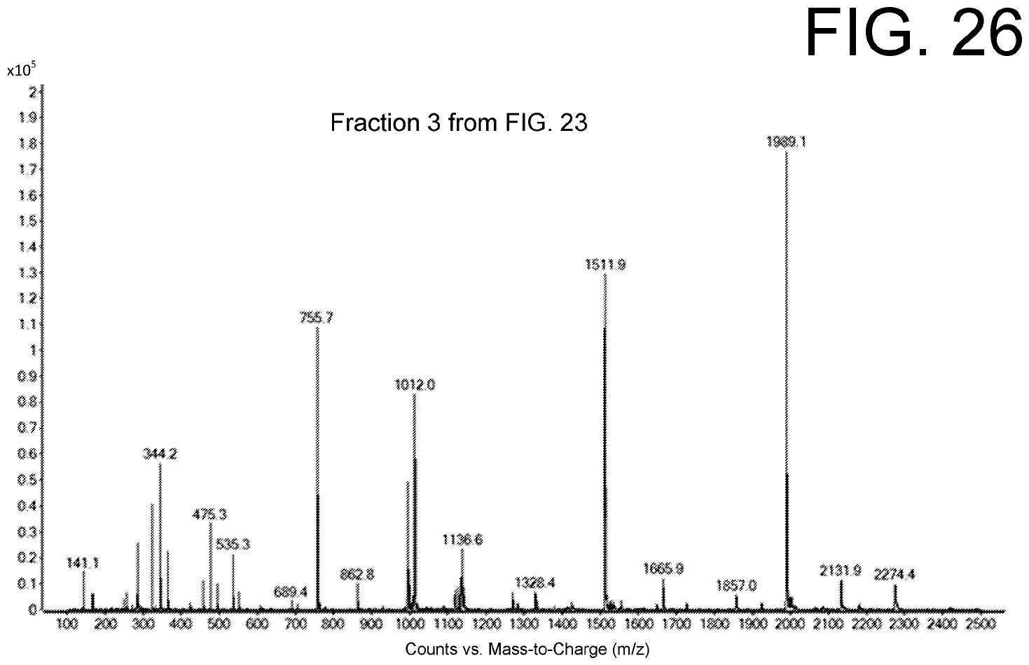

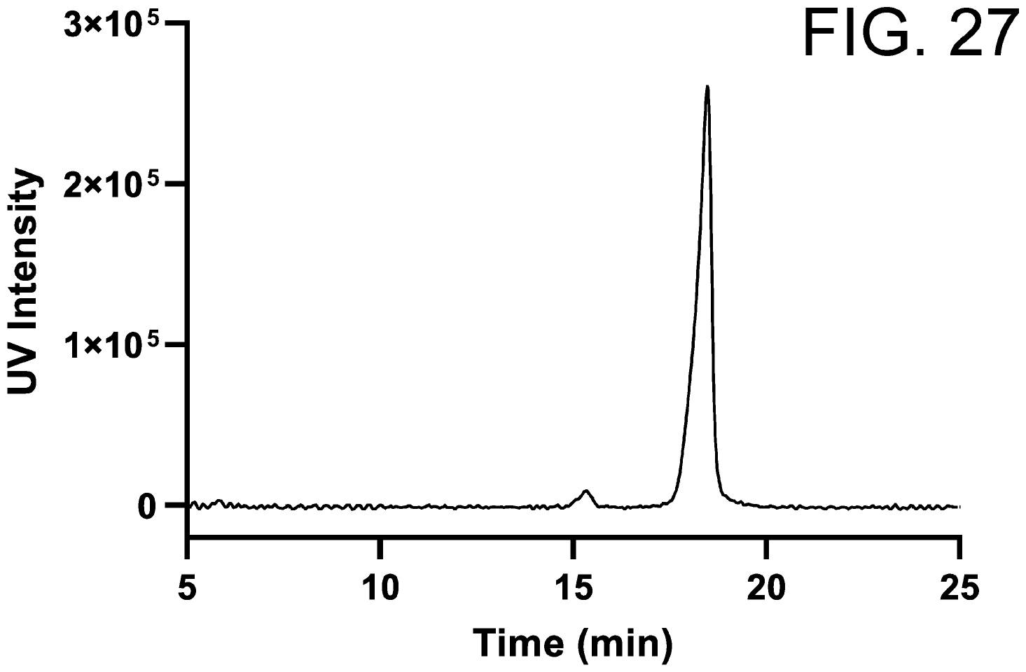

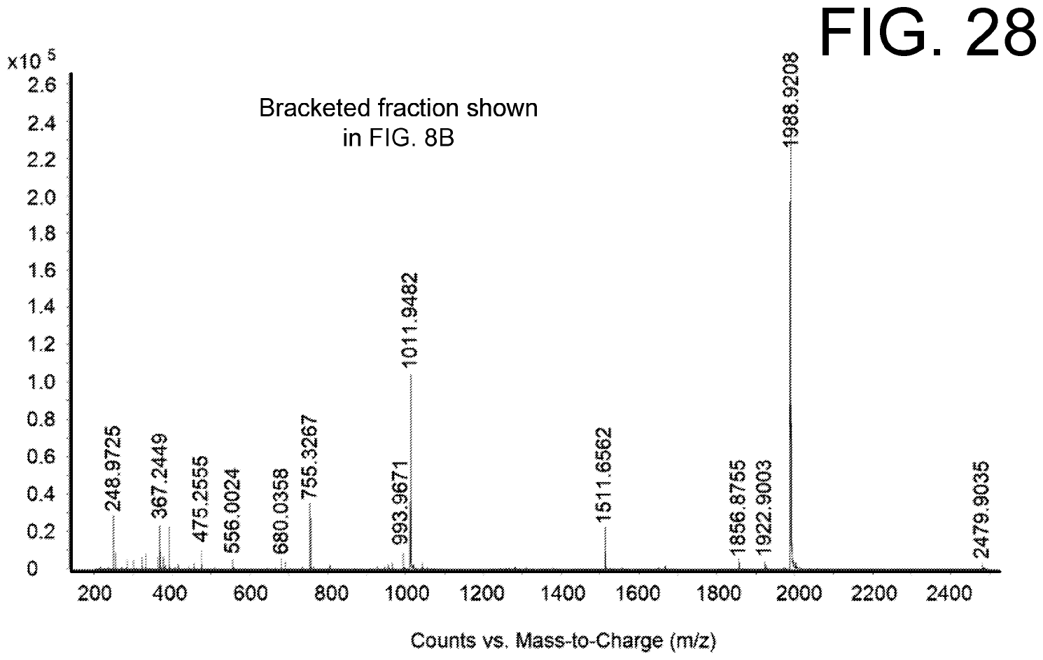

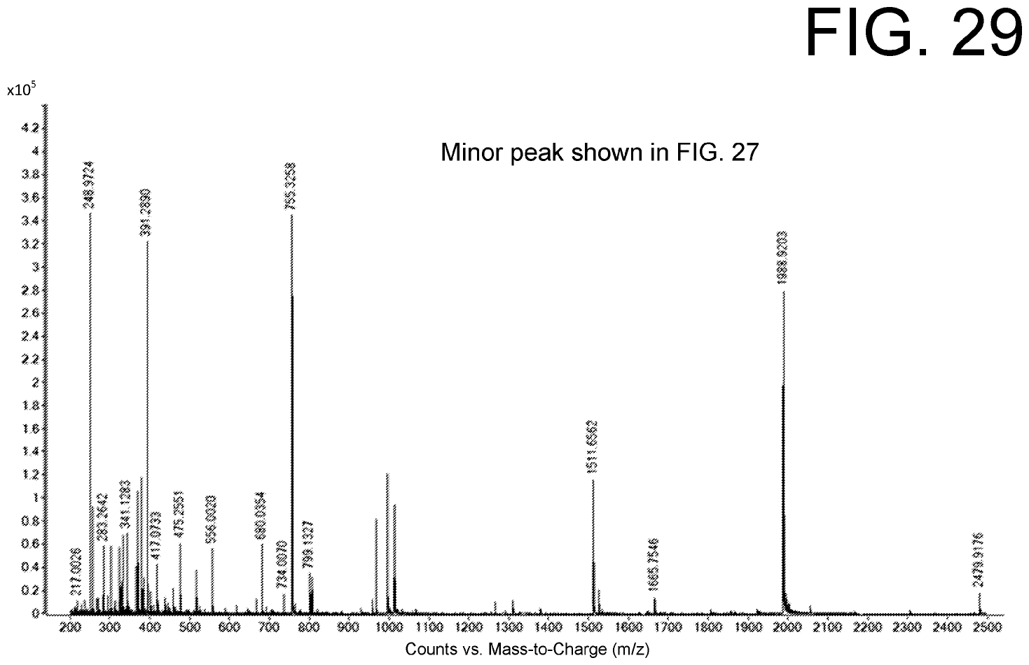

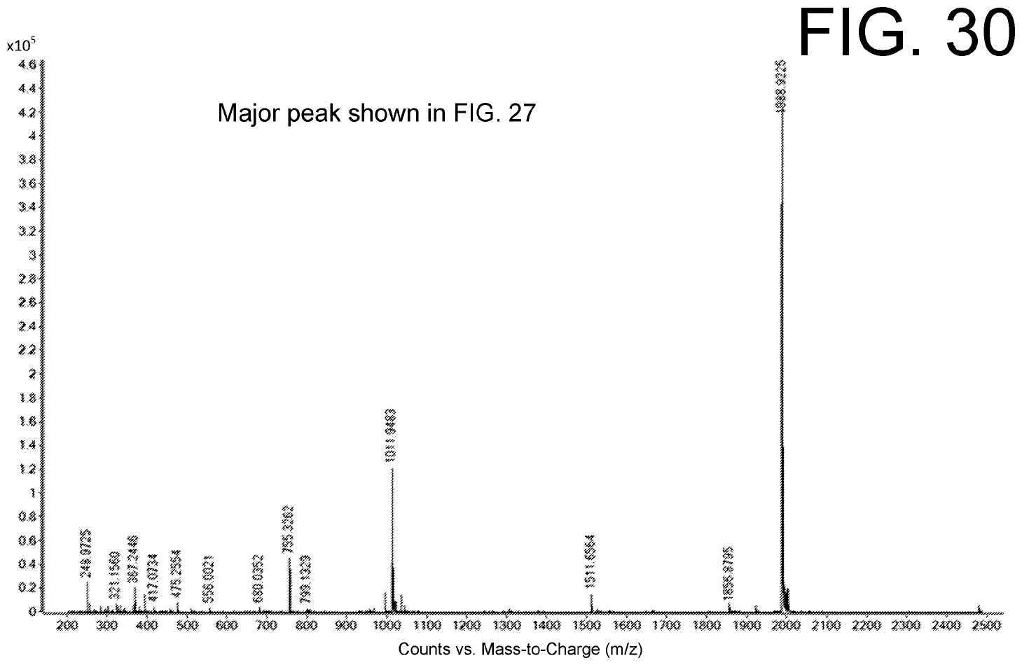

Both the Synergi Hydro RP column and the Luna Omega Polar C18 column outperformed the Gemini C18 column. The Luna Omega Polar C18 column showed the highest resolution. The Luna Omega Polar C18 column is used in the other examples of this disclosure. However, the Synergi Hydro RP column or any other polar RP column is expected to yield similar results. The Luna Omega Polar C18 column is used to create the chromatograms shown in FIG. 2 (solid line), FIG. 6, FIG. 3, and FIG. 4.

### Example 2—Variation of Mobile Phase Condition for Polar RP Column

The separation gradient (mobile phase A: H₂O with 0.1% formic acid, mobile phase B: MeCN with 0.1% formic acid) used on the analytical Luna Omega Polar C18 column in FIG. 1 was progressively narrowed from 20-80% (dot dash line) to 30-70% (dashed line) and then to 40-50% (solid line) of mobile phase B. FIG. 2 shows narrowing of the gradient to 40-55% improved separation of the peak containing QS-21 (indicated by an arrow).

FIG. 3 shows the effects of progressively narrowing the separation gradient on elution time on a preparative Luna Omega Polar C18 (250×50 mm, 5 μm, 100 Å) column. The separation gradient was progressively narrowed from 35-55% (solid line), to 37-50% (dashed line), 38-49% (dotted line), and 40-47% (dot dash line) mobile phase B (MeCN with 0.1% formic acid), in order to move the QS-21-containing peak (indicated by an arrow) to an earlier elution time which reduces run time and solvent consumption. The narrowest separation gradient of 40-47% mobile phase B resulted in the shortest run time of about 27 minutes. The separation gradient of 40-47% mobile phase B was selected for testing of sample loading volumes.

### Example 3—Variation of Sample Loading Volumes for Polar RP Column

FIG. 4 shows the testing of sample volumes on a polar RP column. A preparative Luna Omega Polar C18 column (250×50 mm, 5 μm, 100 Å with a 15 mm length guard column) was loaded with either 5 mL (solid line) or 10 mL (dashed line) of VET-SAP® at 100 mg/mL in H₂O and run with a 40-47% mobile phase B gradient. The 10 mL injection run (dashed line) was fractionated at 0.25 min/fraction and individual fractions were then analyzed by LC/Q-TOF MS in negative ion mode to identify QS-21 content. The area of interest to be collected as an intermediate purity saponin extract includes the individual fractions containing singly charged QS-21 (m/z 1989) as the most abundant ion above m/z 1200 among singly charged, intact saponins. The dotted line trace is a 40-45% mobile phase B gradient with 10 mL sample injection. It is a modification of the dashed line trace where the separation gradient is truncated at 45% mobile phase B after elution of the peak containing QS-21 to further reduce run time and solvent use. This was the condition used for sample processing below and the fraction collection window is indicated on the chromatogram by dotted lines and is referred to as a “polar RP intermediate purity saponin extract.”

The collected fractions containing the polar-RP intermediate purity saponin extract were subjected to a steady stream of nitrogen or filtered air to evaporate the acetonitrile and were then lyophilized to a dry powder. A total of 16.7 g of powdered VET-SAP® was processed which produced 648 mg of lyophilized polar-RP intermediate purity saponin extract for a yield of about 3.9% with a purity of about 50% based on weight of the dried intermediate purity saponin extract as compared to weight of the dried substantially pure QS-21.

### Example 4—Comparison of Intermediate Purity Saponin Extract Generated by a Polar RP Column and a Traditional RP Column

Increased purity of the fraction collected from a polar RP column as compared to a traditional RP column is shown by FIG. 5-FIG. 8. FIG. 5 shows the separation provided by a traditional RP Gemini C18 column. The fraction that was collected for further processing is bracketed by dotted lines. This fraction was identified by negative ion mode LC/single quadrupole MS detection of singly charged QS-21 at m/z 1989 as the most abundant ion above m/z 1200 among singly charged, intact saponins. This fraction is referred to as a traditional-RP intermediate purity saponin extract.

FIG. 6 shows separation provided by a polar RP column. The fraction corresponding to the peak containing QS-21 excluding left and right shoulder peaks, indicated by dotted lines, was collected as a polar RP intermediate purity saponin extract.

FIG. 7 and FIG. 8 show negative ion mass spectra of the traditional-RP intermediate purity saponin extract and polar RP intermediate purity saponin extract, respectively. The mass spectrum shown in FIG. 7 includes considerably more impurities than the mass spectrum shown in FIG. 8, indicating that polar RP chromatography is more effective at purifying partially enriched Quillaja saponaria extracts than traditional RP polar chromatography. FIG. 8 includes a peak containing singly deprotonated QS-21 as the most abundant ion above m/z 1200, with an m/z value of 1,989. The increased purity is evident in the reduced number of impurity ion peaks, particularly at m/z greater than 1,200, where the singly charged saponins are found. (H. L. Pham, B. P. Ross, R. P. McGeary, P. N. Shaw, A. K. Hewavitharana, N. M. Davies, Saponins from Quillaja saponaria Molina: isolation, characterization and ability to form immuno stimulatory complexes (ISCOMs), Curr. Drug Deliv., 3 (2006) 389-397.)

Without being bound by theory, it is believed that the improved purity is attributed at least in part to the increased peak resolution provided by the polar RP column shown in FIG. 6. Increased purity of the intermediate purity saponin extract obtained from the polar RP column as compared to a traditional RP column results in higher purity and yield in the substantially pure saponin extract as less tradeoff is needed between the two parameters.

### Example 5—Purification of a Traditional-RP Intermediate Purity Saponin Extract by Traditional RP Chromatography on a C8 Column

FIG. 9 is a chromatogram showing separation of the traditional-RP intermediate purity saponin extract indicated by the bracketed fraction in FIG. 5 and the mass spectrum in FIG. 7 on a preparative Polaris C8 column (250×21.2 mm, 5 μm, 110 Å). Thus, FIG. 9 shows the elution profile of VET-SAP® separated first on a traditional RP C18 column followed by separation on a traditional RP C8 column. These two chromatographic techniques are not orthogonal to each other because they are both traditional RP chromatography columns. Thus, this is purification using two sequential traditional RP chromatography separations. Use of a traditional RP C8 column in the second step provided minimal additional separation.

The eluate is fractionated and fractions containing singly deprotonated QS-21 as the most abundant ion above m/z 1200 in LC/single quadrupole analysis are collected. The fractions collected as the final purified product, referred to here as dual RP chromatography purified QS-21, are bracketed by the dotted lines.

The yield of lyophilized QS-21 collected after purification on two traditional RP chromatography columns is about 0.5% by weight of the total weight of powdered VET-SAP® used as a starting material. The purity is about 85% (data not shown). Without being bound by theory, it is believed that the yield and purity are limited due to poor chromatographic separation because the second separation step on a traditional RP C8 column provides little additional separation beyond the traditional RP C18 column.

### Example 6—Separation of Intermediate Purity Saponin Extracts on an Unbonded Silica HILIC Column

FIG. 10 shows the results of testing a HILIC column with a unbonded silica solid phase. A Kinetex HILIC column (250×4.6 mm, 5 μm, 100 Å) was tested for the separation of both traditional-RP intermediate purity saponin extract (i.e., fraction indicated in FIG. 5; solid line) and dual RP chromatography purified QS-21 (i.e., fraction indicated in FIG. 9; dashed line). A flat baseline was achieved with this column. Both samples were run isocratically in 80:20 MeCN:H₂O buffered by 5 mM ammonium acetate (pH 5.8). The dual RP chromatography purified QS-21 sample (dashed line) was purified by two rather than one traditional RP chromatography steps resulting in removal of the side peaks shown in the solid line.

### Example 7—Variation of Mobile Phase on an Unbonded Silica HILIC Column

FIG. 11 shows testing of the Kinetex HILIC column used in FIG. 10 buffered at pH 3.2 with ammonium formate (dashed line) compared to pH 5.8 with ammonium acetate (solid line). The lower pH resulted in a later elution time and slightly broadened peaks without significant change to the separation profile.

### Example 8—Separation of Saponin Extracts on an Amide HILIC Column

FIG. 12 shows the results of testing a HILIC column with an amide-functionalized solid phase. A TSKgel-Amide 80 (250×4.6 mm, 5 μm, 100 Å) column was tested for the separation of both traditional-RP intermediate purity saponin extract (i.e., fraction indicated in FIG. 5; solid line) and dual RP chromatography purified QS-21 (i.e., fraction indicated in FIG. 9; dashed line). Both samples were run isocratically in 80:20 MeCN:H₂O buffered by 5 mM ammonium acetate (pH 5.8). A flat baseline was not easily achieved with this column. As is evident from FIG. 12, a cluster of impurity peaks centered around 16 minutes could be baseline separated from the main peak. The ability to separate these impurity peaks in the main peak indicates an improvement using an amide solid phase HILIC column as compared to a unbonded silica HILIC column as shown in FIG. 10.

### Example 9—Separation of Saponin Extracts on Amino HILIC Columns

FIG. 13 shows the results of testing a first HILIC column with an amino-functionalized solid phase. A Zorbax NH₂ (250×4.6 mm, 5 μm, 70 Å) column was tested for the separation of both traditional-RP intermediate purity saponin extract (i.e., fraction indicated in FIG. 5; solid line) and dual RP chromatography purified QS-21 (i.e., fraction indicated in FIG. 9; dashed line). Both samples were run isocratically in 60:40 MeCN:H₂O buffered by 5 mM ammonium acetate (pH 5.8). A flat baseline was not easily achieved with this column. The samples, in particular the traditional-RP intermediate purity saponin extract (solid line), eluted with several impurity shoulder peaks in front of the main peak.

FIG. 15 shows the results of testing a second HILIC column with an amino-functionalized solid phase. A Luna NH₂ (250×4.6 mm, 5 μm, 100 Å) column was used to separate traditional-RP intermediate purity saponin extract isocratically in 50:50 MeCN:H₂O buffered by 5 mM ammonium acetate (pH 5.8). A flat baseline was not easily achieved with this column. This column shows a similar separation profile as the Zorbax NH₂ column shown in FIG. 13. Other amino-based HILIC columns are expected to produce similar separation profiles.

### Example 10—Variation of Mobile Phase Condition for Amino HILIC Chromatography

FIG. 14 shows the effects of varying the isocratic mobile phase conditions on the Zorbax NH₂ column used in FIG. 13. Testing with 50:50 (solid line), 60:40 (dashed line), or 65:35 (dot dash line) of MeCN:H₂O buffered by 5 mM ammonium acetate (pH 5.8) shows that decreasing water content in the mobile phase results in more prominent impurity separation and longer elution time.

### Example 11—Separation of Saponin Extracts on an Amide/Amino HILIC Column

FIG. 18 shows the results of testing a hybrid amide/amino-functionalized HILIC column. A Luna Omega SUGAR (250×4.6 mm, 3 μm, 100 Å) column was tested for the separation of traditional-RP intermediate purity saponin extract (i.e., fraction indicated in FIG. 5) with a mobile phase of 70:30 (solid line) or 75:25 (dashed line) MeCN:H₂O, 5 mM ammonium acetate (pH 5.8). As evident from the chromatograms, this hybrid column chemistry does not appear to offer better separation than either an amide-based HILIC column (FIG. 12) or an amino-based HILIC column (FIG. 13 and FIG. 15) in this particular application.

### Example 12—Separation of Saponin Extracts on a Zwitterionic HILIC Column

FIG. 16 shows the results of testing a HILIC column with a solid phase surface modified with zwitterionic sulfobetaine groups. An analytical Syncronis HILIC (250×4.6 mm, 5 μm, 100 Å) column was tested for the separation of both traditional-RP intermediate purity saponin extract (i.e., fraction indicated in FIG. 5; solid line) and dual RP chromatography purified QS-21 (i.e., fraction indicated in FIG. 9; dashed line). Both samples were run isocratically in 80:20 MeCN:H₂O water buffered with 5 mM ammonium formate (pH 3.2). Several impurity peaks are visible with a baseline-separated cluster centered around 12.5 minutes and others manifested as shoulder peaks.

FIG. 17 shows the results of testing the same Syncronis HILIC column as in FIG. 16 with a change to the buffer and pH of the mobile phase. The column was tested for the separation of both traditional-RP intermediate purity saponin extract (i.e., fraction indicated in FIG. 5; solid line) and dual RP chromatography purified QS-21 (i.e., fraction indicated in FIG. 9; dashed line). Both samples were run isocratically in 80:20 MeCN:H₂O water buffered with 5 mM ammonium acetate (pH 5.8). Changing the buffer to ammonium acetate (pH 5.8) resulted in higher peak resolution as compare to the use of ammonium formate (pH 3.2).

Among all of the HILIC column and condition combinations tested, the Syncronis HILIC column run isocratically in 80:20 MeCN:H₂O, 5 mM ammonium acetate (pH 5.8) showed the best resolution and peak shape. The Syncronis HILIC column also offered one of the shortest run times, with all major peaks eluting before 16 min (FIG. 17), which results in higher throughput due to shorter runtimes and reduced consumption of mobile phase reagents.

The variation in the separation characteristics of the various types of HILIC columns as well as the influence of the elution conditions was not predictable prior to testing. The benefit of following separation on a traditional RP C18 column with separation on a HILIC column rather than a traditional RP C8 column are shown by the improved resolution of all the chromatograms of HILIC separation as compared to FIG. 10.

### Example 13—Comparison of Sample Loading Volumes of Traditional-RP Intermediate Purity Saponin Extract on Zwitterionic HILIC Columns

FIG. 19 shows the results of a loading study performed on the analytical Syncronis HILIC (250×4.6 mm, 5 μm, 100 Å) column used in FIG. 16. Four different injection volumes of 2 (solid line), 5 (dashed line), 30 (dot dash line), and 60 μL (dotted line) of 40 mg/mL traditional-RP intermediate purity saponin extract were tested. Resolution loss occurred between 5 and 30 μL.

FIG. 20 shows the results of a loading study performed on an analytical SeQuant® ZIC®-pHILIC (250×4.6 mm, 5 μm, 200 Å) column. The column was loaded with 5 (dashed line) or 30 (solid line) μL of 40 mg/mL traditional-RP intermediate purity saponin extract and run under the same conditions used to test the Syncronis HILIC column in FIG. 19. Resolution of the SeQuant® ZIC®-pHILIC column was slightly lower than that of the Syncronis HILIC column as shown by comparing the 5 μL (dashed line) trace from FIG. 19 with the 5 μL (dashed line) trace from FIG. 20.

FIG. 21 shows the results of testing two different sample volumes on a preparative zwitterionic Syncronis HILIC column (21.2×250 mm, 5 μm, 100 Å with a 10 mm length guard column). This is a preparative version of the analytical column used in FIG. 16, FIG. 17, and FIG. 19. Samples of traditional-RP intermediate purity saponin extract were dissolved in 65:35 (v:v) MeCN:H₂O, 5 mM ammonium acetate, pH 5.8. The dissolved sample solution was filtered using a disposable 0.2 μm PTFE membrane syringe filter.

The two injection volumes were 250 μL (dashed line) and 600 μL (solid line) at 40 mg/mL of traditional-RP intermediate purity saponin extract. These injection volumes correspond to 12.5 μL and 30 μL on the smaller analytical Syncronis HILIC column (250×4.6 mm, 5 μm, 100 Å) used to generate the chromatogram shown in FIG. 19. These results are consistent with the results obtained on the analytical version of this column. The 600 μL (solid line) injection volume resulted in noticeable loss in resolution compared to 250 μL (dashed line) injection volume.

### Example 14—Characterization of Fractions from Zwitterionic HILIC Chromatography

FIG. 23 shows a preparative zwitterionic Syncronis HILIC column (21.2×250 mm, 5 μm, 100 Å) used to separate 600 μL at 40 mg/mL of traditional-RP intermediate purity saponin extract. This separation was performed under the same conditions as the 600 μL (solid line) injection volume trace shown in A. Three separate fractions are indicated by dashed lines. Fraction 1 includes the left shoulder centered around 15 minutes. Fraction 2 includes the tallest peak eluting around 16 minutes. Fraction 3 includes the second tallest peak eluting around 17 minutes.

FIG. 24 shows a negative ion mass spectrum of fraction 1 from FIG. 23. This fraction from the left shoulder is rich in impurities as indicated by the multiple peaks besides the various forms of QS-21 including singly charged QS-21 with an m/z value of 1,989, doubly charged de-acylated QS-21 with an m/z value of 755, doubly charged QS-21 with an m/z value of 994.0, ammonium adduct of doubly charged QS-21 with an m/z value of 1012, and singly charged de-acylated QS-21 with an m/z value of 1,512.

FIG. 25 shows a negative ion mass spectrum of fraction 2 from FIG. 23. FIG. 26 shows a mass spectrum of fraction 3 from FIG. 23. Fraction 2 and fraction 3 both contain singly deprotonated QS-21 with an m/z value of 1,989 as the most abundant ion. Both fraction 2 and fraction 3 also contained doubly charged de-acylated QS-21 with an m/z value of 755, doubly charged QS-21 with an m/z value of 994.0, ammonium adduct of doubly charged QS-21 with an m/z value of 1012, and singly charged de-acylated QS-21 with an m/z value of 1,512. De-acylation of QS-21 occurs naturally in aqueous solution via hydrolysis of the ester linkage connecting the fatty acyl chain to the fucose ring and may be reduced by minimizing sample handling in aqueous solution. (J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28). In-source fragmentation during mass spectrometry is also a possible cause for de-acylation.

The two peaks corresponding to fraction 2 and fraction 3 may be compositional isomers of QS-21. Without being bound by theory, it is believed that the two different compositional isomers differ in the terminal glycosyl group in the linear tetrasaccharide chain being either a D-apiose or D-xylose with D-apiose eluting first and D-xylose second. These two compositional isomers of QS-21 have been previously separated by HILIC chromatography and found to have comparable adjuvant activity. (U.S. Pat. No. 5,583,112; Kensil, C. A., S. Soltysik, and D. J. Marciani, Saponin-antigen conjugates and the use thereof 1996, Cambridge Biotech Corporation, Worcester, Mass.: United States; and S. Soltysik, D. A. Bedore, C. R. Kensil, Adjuvant Activity of QS-21 Isomers, Ann. N. Y. Acad. Sci., (1993) 392-395). This speculation is further supported by calculating the ratio of the AUCs of the two tallest peaks shown in FIG. 23, giving roughly 68:32, which is in close agreement with the 65:35 ratio for these two composition isomers previously established by others. (J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28.)

Due to the similar adjuvant activity of these two compositional isomers of QS-21, the two tallest peaks from the chromatogram shown in FIG. 23 corresponding to fractions 2 and 3 do not need to be baseline separated for the purpose of collecting purified QS-21 with adjuvant activity.

### Example 15—Purification of Polar RP Intermediate Purity Saponin Extract on Preparative Zwitterionic HILIC Columns

FIG. 22 shows the results of the same comparison of loading volumes performed in FIG. 21 with a different intermediate purity saponin extract. The preparative zwitterionic Syncronis HILIC column was used to separate the polar RP intermediate purity saponin extract obtained as described in FIG. 4. Lyophilized powder obtained in Example 3 was dissolved in 65:35 (v:v) MeCN:H₂O, 5 mM ammonium acetate, pH 5.8. The dissolved sample solution was filtered using a disposable 0.2 μm PTFE membrane syringe filter (Corning Inc., Corning, NJ, USA). The two injection volumes were 250 μL (dashed line) and 600 μL (solid line) at 40 mg/mL. Thus, the difference between the chromatograms shown in FIG. 21 and FIG. 22 is the purification technique used for the first chromatographic step: traditional RP chromatography or polar RP chromatography respectively.

FIG. 22 has fewer peaks eluting earlier than the main peak as compared to FIG. 21. The earlier eluting peaks indicate the presence of impurities. The loss of those earlier eluting peaks further supports the conclusion that polar RP chromatography is superior to traditional RP chromatography as an initial purification step for partially enriched Quillaja saponaria extracts.

The smaller injection volume, 250 μL (dashed line), exhibited improved separation showing a better resolved peak following the main peak than the larger injection volume of 600 μL (solid line). The two predominant peaks in the both traces are believed to correspond to two compositional QS-21 isomers, where the terminal glycosyl group in the linear tetrasaccharide chain is either a D-apiose or D-xylose. LC/QqQ-MS shows that both peaks contain m/z 1989 as the most abundant ion. The two isomers may be combined in a final purified product due to their demonstrated similar adjuvant activity. (U.S. Pat. No. 5,583,112) Accordingly, any injection volume from about 250-600 μL would be suitable.

Eluate of the 600 μL (solid line) injection was fractionated and the fractions were analyzed by LC/Q-TOF MS to identify the area of interest to be collected as substantially pure QS-21. The fraction collected as substantially pure QS-21 is bracketed by dotted lines. The substantially pure QS-21 was subsequently lyophilized to a dry powder.

All 640 mg of the lyophilized polar RP intermediate purity saponin extract obtained in Example 3 was separated with repeated injections to produce 339 mg of substantially pure QS-21 following lyophilization. This represents a yield of about 2% of the initial 16.7 g of dried VET-SAP®. It is understood that the amount of QS-21 or any other saponins present in VET-SAP® is not consistent batch to batch. The final yield may increase or decrease depending on the amount of QS-21 in the starting material.

### Example 16—Characterization of Substantially Pure QS-21

The lyophilized powder containing substantially pure QS-21 produced as shown in Example 12 (bracket fraction collected in FIG. 22) was characterized both by analytical chromatography and mass spectrometry. The fractions bracketed by the dashed lines in FIG. 22 were lyophilized and resuspended using the procedures indicated above.

FIG. 27 shows UV_{210 nm} trace of 50 μL of substantially pure QS-21 dissolved in 35 v % MeCN with 0.1% formic acid characterized on an analytical Vydac C4 column (250×4.6 mm, 5 μm, 300 Å). There is a minor peak at about 15.5 minutes and a major peak at about 18.0 minutes.

FIG. 28 shows LC/Q-TOF MS characterization of the substantially pure QS-21 collected in FIG. 22 in negative ion mode following lyophilization and subsequent resuspension using the procedures indicated above. This sample includes both the minor peak and the major peak shown in FIG. 27. Singly charged QS-21 with an m/z value of 1989 was detected as the most abundant ion. Three impurity ions at m/z 1,857, 1,923, and 2,480 were detected at much lower intensities than singly charged QS-21. Other ions detected in the mass spectrum include doubly charged QS-21 at m/z 994, ammonium adduct of doubly charged QS-21 at m/z 1,012, singly charged de-acylated QS-21 at m/z 1,512, and doubly charged de-acylated QS-21 at m/z 755. Notably, the purification techniques of this disclosure removed the impurity ion with m/z 2,018 that was not able to be removed by the saponin purification techniques described in WO 2019/106192.

LC/MS analysis was performed to elucidate the compositions of the two peaks present in the analytical chromatogram. FIG. 29 shows characterization of the minor peak from FIG. 27 that elutes at about 15.5 minutes by LC/Q-TOF MS in negative ion mode. Singly charged QS-21 with an m/z value of 1989 was detected as the most abundant ion above m/z 1200.

FIG. 30 shows characterization of the major peak from FIG. 27 that elutes at about 18.5 minutes by LC/Q-TOF MS. This mass spectrum is similar to the mass spectrum shown in FIG. 28. This is expected because about 97% of the combined sample is the major peak while the minor peak contributes only about 2%. Thus, excluding the minor peak of FIG. 27 had only a slight effect on purity.

LC/Q-TOF MS revealed that QS-21 appears as the most abundant ion above m/z 1200 in both the major and minor peaks (FIG. S7B and FIG. S7C). Without being bound by theory, it is believed that the two peaks correspond to A- and B-regioisomers of QS-21, respectively, which naturally occur in solution from the intramolecular trans-esterification of the acyl chain between the 3- and 4-hydroxyl groups on the fucose ring. (N. E. Jacobsen, W. J. Fairbrother, C. R. Kensil, A. Lim, D. A. Wheeler, M. F. Powell, Structure of the saponin adjuvant QS-21 and its base-catalyzed isomerization product by 1H and natural abundance 13C NMR spectroscopy, Carbohydr. Res., 280 (1996) 1-14; Y. Wang, X. Lu, G. Xu, Development of a comprehensive two-dimensional hydrophilic interaction chromatography/quadrupole time-of-flight mass spectrometry system and its application in separation and identification of saponins from Quillaja saponaria, J. Chromatogr. A, 1181 (2008) 51-59; J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28; C. Pedebos, L. Pol-Fachin, R. Pons, C. V. Teixeira, H. Verli, Atomic model and micelle dynamics of QS-21 saponin, Molecules, 19 (2014) 3744-3760.) This interconversion can likely be reduced by minimizing sample processing in aqueous solution, but the minor peak does not need to be removed as impurity because previous work has separated the two regioisomers on a C4 column and confirmed that both have adjuvant activity. (J. L. Cleland, C. R. Kensil, A. Lim, N. E. Jacobsen, M. S. L. Basa, D. A. Wheeler, J.-Y. Wu, M. F. Powell, Isomerization and Formulation Stability of the Vaccine Adjuvant QS-21, J. Pharm. Sci., 85 (1996) 22-28.)

The percent area under the curve (AUC) of the major peak in FIG. 27 was calculated to be slightly greater than 97% of the total area, suggesting that the final purified product, excluding the B-regioisomer, is at least 97% pure. Purification by two sequential traditional RP chromatography steps (C18 column followed by C8 column) produced a final sample with only 85% purity as identified by the same calculation of AUC of the major peak (data not shown).

### CONCLUSION

Although the subject matter has been described in language specific to features and/or methodological acts, it is to be understood that the subject matter defined in the appended claims is not necessarily limited to the specific features or acts described above. Rather, the specific features and acts are disclosed as example forms of implementing the claims.

Certain implementations are described herein, including the best mode known to the inventors for carrying out the invention. Of course, variations on these described implementations will become apparent to those of ordinary skill in the art upon reading the foregoing description. Skilled artisans will know how to employ such variations as appropriate, and the implementations disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

1. A substantially pure saponin extract derived from Quillaja saponaria comprising QS-21 characterized by a purity of at least 95% and mass spectrum indicating a most abundant species at mass-to-charge ratio (m/z) 1989 and impurities at m/z 1857, 1923, and 2480 in negative ion mode.

2. The substantially pure saponin extract of claim 1, further characterized by the absence of other impurity mass spectral peaks between m/z 750-2500 with an intensity greater than one-third the intensity of the m/z 1989 peak.

3. The substantially pure saponin extract of any of claims 1-2, further characterized by the absence of a mass spectral peak at m/z 2018.

4. The substantially pure saponin extract of any of claims 1-3, wherein the purity is at least 97%.

5. The substantially pure saponin extract of any of claims 1-4, wherein the purity is measured by area under the curve (AUC) of an analytical chromatography ultraviolet (UV) trace.

6. The substantially pure saponin extract of claim 5, wherein the analytical chromatography is performed with a traditional reversed-phase (RP) C4 column comprising a butyl-functionalized silica solid phase and a water/acetonitrile mobile phase including a volatile modifier.

7. The substantially pure saponin extract of any of claims 5-6, wherein the UV trace is characterized by a minor peak preceding a major peak and purity is measured by the percent AUC of the of the major peak.

8. The substantially pure saponin extract of any of claims 1-7, further characterized by adjuvant activity.

9. A pharmaceutical composition comprising the substantially pure saponin extract of any of claims 1-8.

10. The pharmaceutical composition of claim 9, further comprising an antigen.

11. A vaccine composition comprising an antigen and the substantially pure saponin extract of any of claims 1-8.

12. Use of the substantially pure saponin extract of any of claims 1-8 in the manufacture of a medicament.

13. A method of eliciting or enhancing an immune response in a subject, the method comprising administering to the subject the substantially pure saponin extract of any of claims 1-8, the pharmaceutical composition of any of claims 9-10, or the vaccine composition of claim 11 in an amount sufficient to elicit or enhance the immune response of the subject.

14. A method of saponin purification comprising:
purifying a partially enriched Quillaja saponaria extract by reversed-phase chromatography to create an intermediate purity saponin extract; and
purifying the intermediate purity saponin extract by hydrophilic interaction liquid chromatography (HILIC) to create a substantially pure saponin extract.

15. The method of claim 14, wherein a saponin in the substantially pure saponin extract is QS-7, QS-17, QS-18, or QS-21.

16. The method of any of claims 14-15, further comprising removing solvent from the intermediate purity saponin extract to provide a dried saponin extract and resuspending the dried saponin extract, wherein the resuspension of the dried saponin extract is provided as the intermediate purity saponin extract for purification by HILIC.

17. The method of any of claims 14-15, further comprising collecting a fraction from the reversed-phase chromatography eluate containing the saponin, wherein the intermediate purity saponin extract is the fraction.

18. The method of claim of 17, wherein the fraction is characterized by negative ion mass spectrum indicating a most abundant species above m/z 1200 among singly charged, intact saponins at m/z 1989.

19. The method of claim of 17, wherein the fraction is characterized by negative ion mass spectrum indicating a most abundant species above m/z 1200 among singly charged saponins at m/z 1862.

20. The method of claim of 17, wherein the fraction is characterized by negative ion mass spectrum indicating a most abundant species above m/z 1200 among singly charged saponins at m/z 2296.

21. The method of claim of 17, wherein the fraction is characterized by negative ion mass spectrum indicating a most abundant species above m/z 1200 among singly charged saponins at m/z 2150.

22. The method of claim 17, wherein the fraction does not include shoulder peaks.

23. The method of any of claims 14-22, further comprising removing solvent from the substantially pure saponin extract to provide a dried saponin extract.

24. The method of claim 23, wherein removing the solvent comprises lyophilization.

25. The method of any of claims 14-24 wherein the reversed-phase chromatography is traditional reversed-phase chromatography.

26. The method of claim 25, wherein the traditional reversed-phase chromatography is performed using a C18 column.

27. The method of any of claims 14-24 wherein the reversed-phase chromatography is polar reversed-phase chromatography.

28. The method of any of claim 27, wherein the polar reversed-phase chromatography is performed using a polar C18 column.

29. The method of any of claims 14-28, wherein the reversed-phase chromatography uses a solid phase comprising unbonded or surface-functionalized silica.

30. The method of any of claims 14-28, wherein the reversed-phase chromatography uses a solid phase comprising a ceramic or a polymer.

31. The method of any of claims 14-28, wherein the reversed-phase chromatography uses a solid phase with a pore size of about 60-300 Å.

32. The method of any of claims 14-28, wherein the reversed-phase chromatography uses a mobile phase comprising water and an organic solvent.

33. The method of claim 32, wherein the organic solvent comprises acetonitrile, methanol, tetrahydrofuran, ethanol, or isopropanol.

34. The method of any one of claims 32-33, wherein the mobile phase further comprises a volatile modifier.

35. The method of claim 34, wherein the volatile modifier comprises formic acid, trifluoroacetic acid (TFA), or acetic acid.

36. The method of any of claims 14-35, wherein the reversed-phase chromatography is performed under gradient conditions.

37. The method of claim 36, wherein the gradient conditions comprise a stepped gradient.

38. The method of claim 36, wherein the gradient conditions comprise a continuous gradient.

39. The method of any of claims 36-38, wherein the gradient conditions are 20-80% organic solvent.

40. The method of any of claim 36-38, wherein the gradient conditions are 30-70% organic solvent.

41. The method of any of claim 36-38, wherein the gradient conditions are 40-50% organic solvent.

42. The method of any of claims 14-35, wherein the reversed-phase chromatography is performed under isocratic conditions.

43. The method of any of claims 14-42, wherein the HILIC uses a solid phase comprising unbonded or surface-functionalized silica.

44. The method of any of claims 14-42, wherein the HILIC uses a solid phase comprising a ceramic or a polymer.

45. The method of any of claims 14-44, wherein the HILIC uses a solid phase surface-functionalized with diol functional groups.

46. The method of any of claims 14-44, wherein the HILIC uses a solid phase surface-functionalized with amide groups.

47. The method of any of claims 14-44, wherein the HILIC uses a solid phase surface-functionalized with amino groups.

48. The method of any of claims 14-44, wherein the HILIC uses a solid phase surface-functionalized with amide groups and amino groups.

49. The method of any of claims 14-44, wherein the HILIC uses a solid phase surface-functionalized with zwitterionic groups.

50. The method of any of claims 14-49, wherein the HILIC uses a solid phase with a pore size of about 70-200 Å.

51. The method of any of claims 14-50, wherein the HILIC uses a mobile phase comprising water and an organic solvent.

52. The method of claim 51, wherein the organic solvent comprises acetonitrile, acetone, methanol, or isopropanol.

53. The method of any of claims 51-52, wherein the mobile phase further comprises a volatile salt.

54. The method of claim 53, when the volatile salt comprises ammonium acetate or ammonium formate.

55. The method of any of claims 14-54, wherein the HILIC is performed under isocratic conditions.

56. The method of claim 55, wherein the mobile phase comprises about 10%, 20%, 30%, 40%, or about 50% water.

57. The method of any of claims 14-54, wherein the HILIC is performed under gradient conditions.

58. The method of claim 57, wherein the mobile phase comprises between about 10-50% water.

59. The method of any of claims 14-58, wherein the HILIC is performed at about pH 5-6.

60. The method of any of claim 59, wherein the HILIC is performed at about pH 5.8.

61. The method of any of claims 14-60, wherein the intermediate purity saponin extract is characterized by a purity of about 50% and negative ion mass spectrum indicating a most abundant species above mass-to-charge ratios (m/z) 1200 among singly charged, intact saponins at m/z 1989.

62. The method of any of claims 14-61, wherein the substantially pure saponin extract is characterized by mass spectral peaks indicating a most abundant species at mass-to-charge ratio (m/z) 1989 and impurities at m/z 1857, 1923, and 2480.

63. The method of any of claims 14-61, wherein the substantially pure saponin extract is characterized by a purity of at least 95% as measured by area-under-the-curve (AUC) of an analytical chromatography UV trace.
